(19) United States
(12) Patent Application Publication
(10) Pub. No.: US 2012/0221110 A1
(43) Pub. Date: Aug. 30, 2012

(54) ARTIFICIAL JOINT

(76) Inventors: Yoshitaka Nakanishi, Kumamoto-shi (JP); Mutsumi Touge, Kumamoto-shi (JP)

(21) Appl. No.: 13/502,380

(22) PCT Filed: Oct. 21, 2010
(86) PCT No.: PCT/JP2010/068616

§ 371 Date: Apr. 17, 2012

(30) Foreign Application Priority Data
Oct. 23, 2009 (JP) 2009-244082
Mar. 17, 2010 (JP) 2010-060129

(51) Int. Cl. A61F 2/30

(52) U.S. Cl. 623/18.11

(57) ABSTRACT

An artificial joint includes a ball and a cup constituting a pair of joint members which forms a joint. A convex curved surface and a concave curved surface which constitute a pair of friction surfaces which slides relative to each other are provided between the pair of joint members. The convex curved surface which is at least one friction surface out of the convex curved surface and the concave curved surface includes: at least either one of groove-shaped recessed portions and hole-shaped recessed portions having a width gradually narrowed toward the inside of the convex curved surface from a surface side of the convex curved surface; and curved surface portions which smoothly connect inclined surface portions which form the recessed portions and a surface which forms a surface portion of the convex curved surface.

### CROSS REFERENCE TO RELATED APPLICATIONS

This application relates to and claims priority from PCT/JP2010/068616, filed on Oct. 21, 2010, the entire contents of which are incorporated herein fully by reference, which in turn relates to and claims priority from JP Patent Applications JP2009-244082 filed on Oct. 23, 2009 and JP2010-060129 filed on Mar. 17, 2010.

### FIGURE SELECTED FOR PUBLICATION

FIG. 5

### BACKGROUND

1. Field of the Invention

The present invention relates to an artificial joint, and more particularly to a shape of a friction surface (slide surface) which an artificial joint has.

2. Description of the Related Art

An artificial joint has friction surfaces (slide surfaces) which slide relative to each other in a state where the friction surfaces come into contact with each other. To be more specific, the artificial joint includes a pair of joint members which is connected to each other so as to form a joint. Contact surfaces at a connecting portion between the joint members form the friction surfaces which slide relative to each other with a lubrication fluid therebetween along with the movement of the joint. As the combination of friction materials which form the friction surfaces in the artificial joint, it is often the case where the combination of a resin material such as polyethylene and a hard material such as metal or ceramics is selected.

Accordingly, with respect to the friction surface of the artificial joint, wear (abrasion) of the friction material on a soft resin side relative to a hard material becomes a problem. To cope with this problem, conventionally, there have been taken measures such as lowering of surface roughness of the hard material or the improvement of hydrophilic and hydrophobic properties on the friction surfaces. Further, although priority of alumina-based or zirconia-based ceramics has been proved through examples with respect to the hard material, a rupture problem attributed to a property of these materials that toughness is low arises as a problem in an actual use. From this point of view, with respect to the hard material, a failsafe property of a metallic material having high toughness and ductility should not be overlooked.

Particularly, an artificial hip joint which is one type of artificial joints adopts, in general, the connection structure where a ball-shaped portion forming a convex curved surface is fitted in a cup-shaped portion having a concave curved surface and hence, shapes of friction surfaces are simple. Accordingly, in the artificial hip joint, an attempt has been made to suppress the wear by optimally adjusting a size of a gap in the radial direction formed between a concave curved surface and a convex curved surface which become friction surfaces, surface roughness of the friction surfaces or the like (see JP-T-2000-508212 (patent document 1), for example). However, in the artificial hip joint, as an actual problem, it is often the case where a fluid film formed of a lubrication fluid between the friction surfaces is broken when a user of the artificial hip joint stands for a long time or the like. Accordingly, it is necessary to improve a lubrication property of the friction surface and the speedy reformation of the fluid film.

Further, as a technique which suppresses the wear of a friction material in an artificial joint, there has been known a technique which forms groove-shaped recessed portions on a friction surface (see JP-A-7-299086 (patent document 2), for example). In the technique disclosed in patent document 2, the recessed portions formed on the friction surface are used as parts for embedding a synthetic resin which forms a thin film as a solid lubrication film on a friction surface on a hard material side or as parts into which wear particles of a resin flows by lubrication oil.

In this manner, in the artificial joint, the suppression of wear of a friction material, particularly a resin material such as polyethylene exists as a task. Conventionally, the wear caused by the friction of a friction material has been a main factor for deciding a lifetime of the artificial joint.

As a countermeasure to cope with the wear of a friction material in an artificial joint, there has been proposed the improvement of properties of a resin material such as polyethylene which is a friction material, the improvement of shape and design of a friction material from a viewpoint of preventing a phenomenon that portions where a contact pressure is high are locally are formed on a friction surface. To improve properties of a resin material, as a specific means, there has been proposed the polymerization of a resin material, cross-linking of a resin material due to the radiation of gamma beams or the like. Further, with respect to the shape and design of the friction material, shape and design which can make a contact pressure on a friction surface uniform and can lower the contact pressure are adopted. By taking such countermeasures to cope with the wear of the friction material, the wear of a friction material in an artificial joint can be suppressed and hence, a drawback that a lifetime of an artificial joint is shortened due to wear caused by the friction of a friction material is almost eliminated currently.

However, due to the above-mentioned countermeasure to cope with the wear, mainly, due to the improvement of properties of a resin material, there arises a phenomenon that wear particles generated from a friction surface of an artificial joint due to the wear becomes small in size. In fact, it is known that wear particles having a size of approximately several μm to sub microns are generated from a friction surface of an artificial joint. Such reduction in size of the wear particles induces a bio-reaction (bioactivity) undesirable in a human body in which an artificial joint is embedded.

To be more specific, minute wear particles made of polyethylene or the like are generated from the friction surface of the artificial joint, the wear particles becomes a subject of a phagocytosis of macrophages. The macrophages which eat wear particles discharge inflammatory cytokine such as TNF-α or IL-6 through the intracellular signal transmission. Inflammatory cytokine which is discharged from macrophages activates osteoblast cells, and generates osteolysis with inflammation around an artificial joint. Osteolysis which is generated around the artificial joint generates loosening of the artificial joint which becomes a cause of an artificial joint replacement operation. The artificial joint replacement operation imposes a large burden on a patient and this burden is particularly large to an aged patient.

With respect to the bioactivity of macrophages brought about by wear particles, it has been found that a size of wear particles largely influences the degree of bioactivity (see J. Bridgest Matthews, Alfred A. Besong. Tim R. Green, Martin H. Stone, B. Mike Wroblewski, John Fisher, Eileen Ingham Evaluation of the Response of Primary Human Peripheral Blood Mononuclear Phagocytes to Challenge with In-Vitro Generated Clinically Relevant UHMWPE Particles of Known Size and Dose, Journal of Biomedical Material Research, 296-307, 2000 (non-patent document 1), for example). According to the studies made in the past or the like, when the size of wear particles becomes smaller than approximately 1 the bioactivity of macrophages becomes conspicuous.

Accordingly, as a method for suppressing the bioactivity of macrophages brought about by wear particles, a method which increases a size of wear particles generated from a friction surface and a method which brings a configuration of wear particles into a configuration where bioactivity is not induced while maintaining a weight of individual wear particles can be considered. As the former method, for example, a plurality of wear particles are coagulated or are integrally formed so that the wear particles are formed into large lumpy particles. Further, as the latter method, for example, a shape of wear particles is formed into an elongated shape having a length which substantially prevents macrophages from eating wear particles or wear particles are formed into a cotton-dust-like shape thus lowering density of wear particles whereby an apparent size of wear particles is set to a value which prevents macrophages from eating wear particles.

In carrying out these methods, the above-mentioned changing properties of a resin material or changing a shape of the friction material is not effective. In fact, to analyze polyethylene and cross-linked polyethylene under the same friction condition, although cross-linked polyethylene is smaller than polyethylene with respect to a size of wear particles, polyethylene and cross-linked polyethylene exhibit the substantially same values with respect to values relating to the configuration of wear particles such as an aspect ratio, circularity or the like of wear particles. Further, also with respect to the relationship between an artificial hip joint and an artificial knee joint which differ in a friction state in view of a surface of a shape of a friction material, in the same manner as the relationship between polyethylene and cross-linked polyethylene, although the artificial hip joint and the artificial knee joint differ in size of wear particles, they exhibit the substantially same values with respect to values relating to the configuration of wear particles.

Further, as a method of increasing a size of wear particles generated from a friction surface, there has been known a method where a friction surface of a hard material made of metal or the like which constitutes a counterpart friction material for a resin material such as polyethylene is made coarse by forming flaws on the friction surface thus increasing a size of wear particles made of a resin material. With respect to such a method which makes the friction surface of a hard material side coarse, there has been reported a result of studies where although the number of or a quantity of wear particles having a size of 0.1 to 1.0 μm which are liable to become a subject of a phagocytosis of macrophages is decreased so that bioactivity of macrophages brought about by an individual wear particle is decreased, the total number of or the total quantity of wear particles is increased approximately several times thus eventually increasing the bioactivity of macrophages as a whole.

Here, the study made on the relevancy between surface roughness of a friction surface and a wear quantity in an artificial joint is added. In view of facts that viscosities of body fluid and a secondary joint fluid which make a gap between friction surfaces wet are low and a load applied between the friction surfaces is large (for example, approximately 3.5 times as large as a body weight), it is safe to say that the environment of friction surfaces of the artificial joint is near a dry state rather than a wet state. Accordingly, when the surface roughness of the friction surface becomes large, the cutting-type wear becomes dominant so that a wear quantity is increased, while when the surface roughness of the friction surface becomes small, the coagulation-type wear becomes dominant so that a wear quantity is increased. That is, in the friction surface environment near a dry state, the relevancy that the smoother the surface of the friction surface, the smaller a wear quantity becomes is not established, and an optimum value range where a wear quantity is decreased exists with respect to the surface roughness of the friction surface. Currently, many artificial joints are manufactured such that the surface roughness of a friction surface on a hard material side falls within a range from 0.02 to 0.2 μm in terms of a Ra value.

As has been explained above, currently, the generation of bio-reaction of macrophages which is brought about by making a size of wear particles finer is considered as a main factor which decides a lifetime of an artificial joint in place of wear caused by the friction of a friction material which has been considered as a main factor conventionally. To suppress the bioactivity of macrophages brought about by wear particles, the improvement of properties of a resin material or a shape of a friction material or the adjustment of surface roughness of a friction surface expressed by an Ra value or the like may be considered. However, the suppression of bioactivity by such improvement or adjustment is limited. Particularly, with respect to the surface roughness of the friction surface, when the environment of the friction surface is in a completely wet state, from a viewpoint of suppressing the wear of the friction material which generates wear particles, the smaller the Ra value, the better the environment of the friction surface becomes. However, because of the reason that, in the artificial joint, viscosity of a body fluid or the like present between the friction surfaces is low as described previously or the like, it is difficult to realize a completely wet environment on the friction surface.

### SUMMARY

The present invention has been made in view of the above-mentioned background, and it is an object of the present invention to provide an artificial joint which can suppress the bioactivity of macrophages brought about by wear particles generated from a friction surface while suppressing the wear of a friction material.

According to one aspect of the present invention, there is provided an artificial joint which includes a pair of joint members which constitutes a joint, and forms a pair of friction surfaces which slides relative to each other between the pair of joint members in a state where the friction surfaces are brought into contact with each other by way of a lubrication fluid, wherein at least one friction surface out of the pair of friction surfaces includes at least either one of groove-shaped recessed portions or hole-shaped recessed portions having a width gradually narrowed from a surface side to the inside of the friction surface, and curved surface portions which smoothly connect surfaces which form the recessed portions and surfaces which form a surface portion of the friction surface.

In one embodiment of the present invention, it is preferable that a depth of the recessed portion is of a sub micron size at largest.

In one embodiment of the present invention, it is preferable that out of the pair of friction surfaces, the friction surface which is formed by one joint member is made of a metallic material, and the friction surface which is formed by the other joint member is made of a resin material (polymer), and the recessed portions and the curved surface portions are formed on the friction surface made of the metallic material.

In one embodiment of the present invention, it is preferable that the recessed portion is used as a shape portion for controlling at least one of a quantity, a size and a shape of wear particles generated from the friction surface by adjusting at least one of a size, a shape and the distribution density of the recessed portions on the friction surface.

In one embodiment of the present invention, it is preferable that the recessed portions are formed such that wear particles generated from the friction surface have a size or a shape by which the wear particles generated from the friction surface are excluded from a subject of a phagocytosis of macrophages.

According to the present invention, it is possible to suppress the bioactivity of macrophages brought about by wear particles generated from a friction surface while suppressing the wear of a friction material.

### BRIEF DESCRIPTION OF THE DRAWINGS

FIG. 1 is a cross-sectional view showing the friction surface structure according to one mode for carrying out the present invention;

FIG. 2 is an explanatory view showing one example of a method of working a friction surface according to one mode for carrying out the present invention;

FIG. 3 is a cross-sectional view showing another example of the friction surface structure according to one mode for carrying out the present invention;

FIG. 4 is a view showing the constitution of an artificial joint according to one mode for carrying out the present invention;

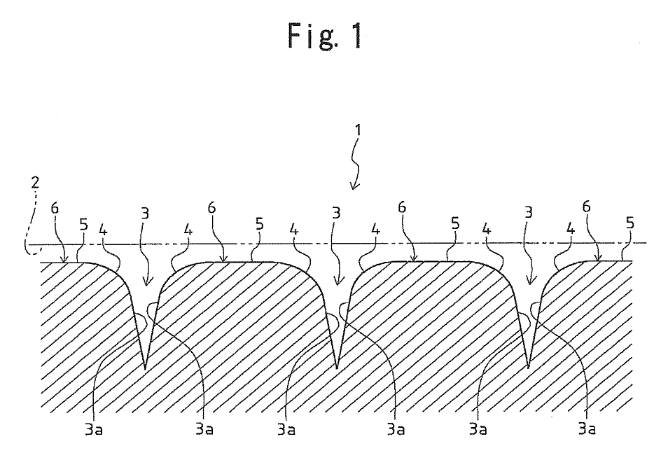

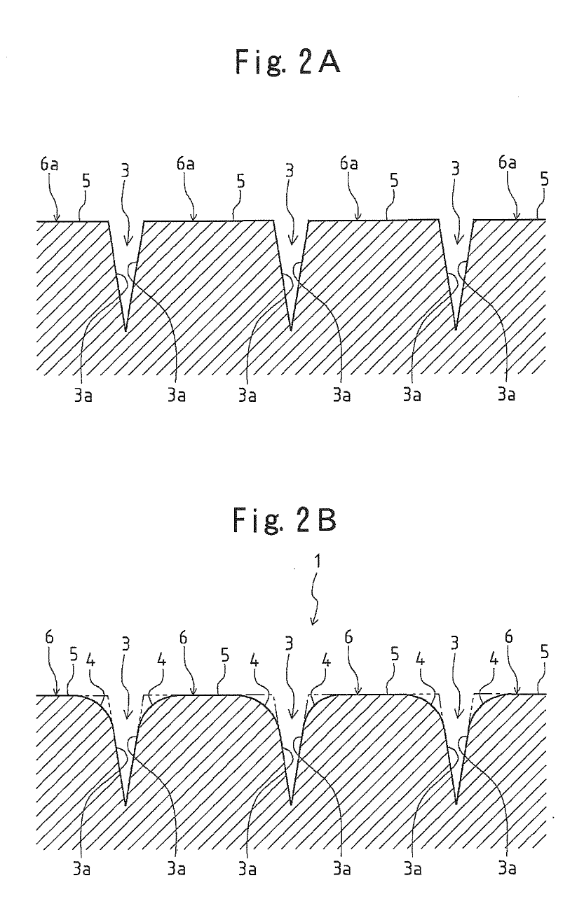

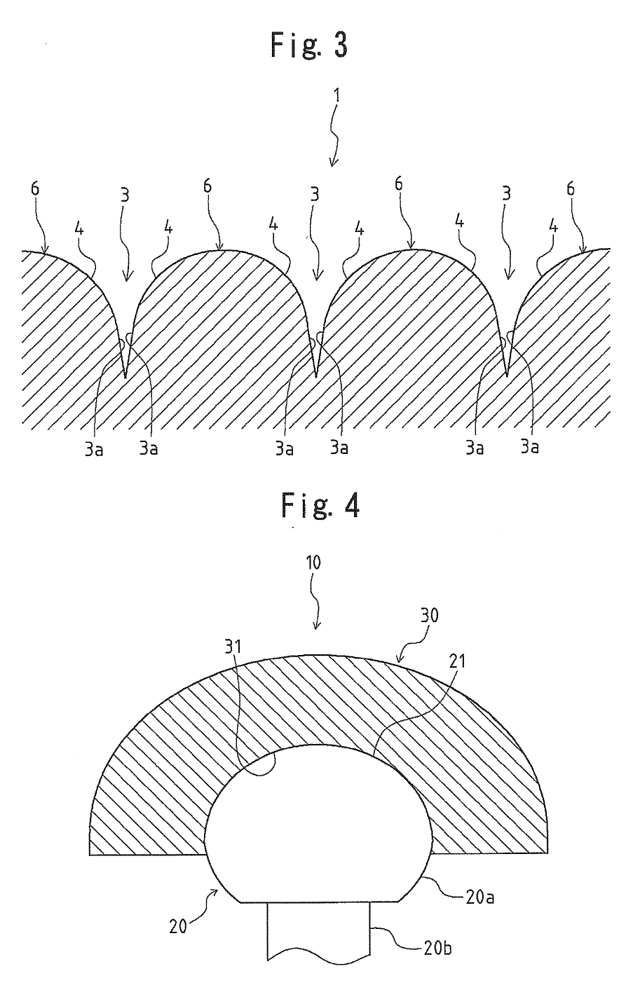

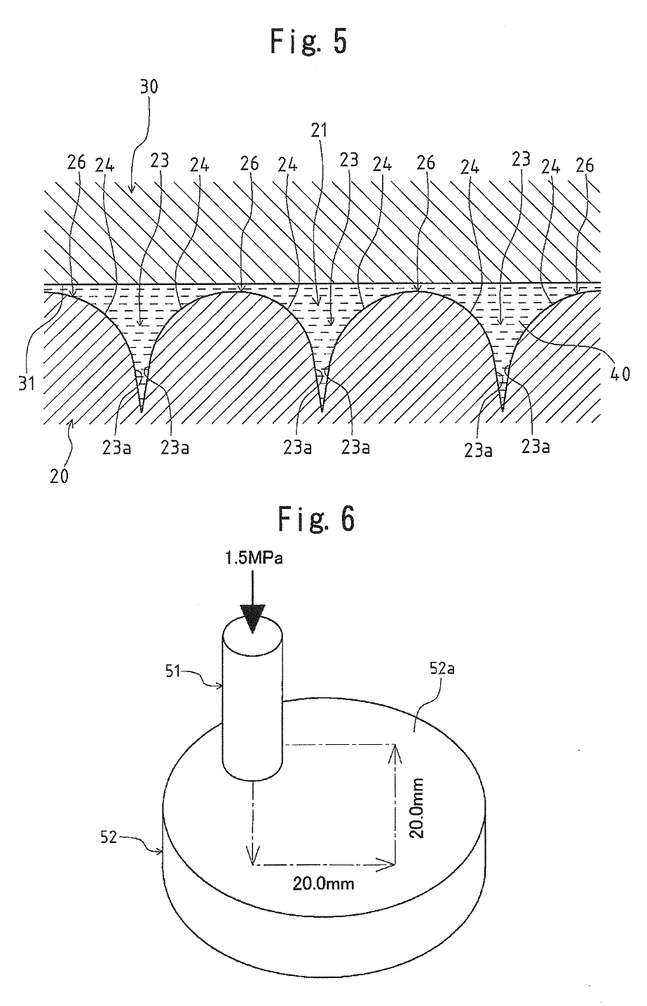

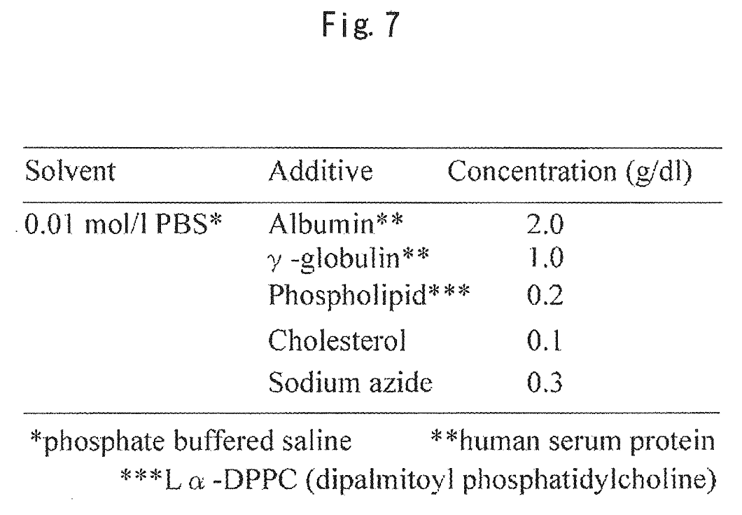

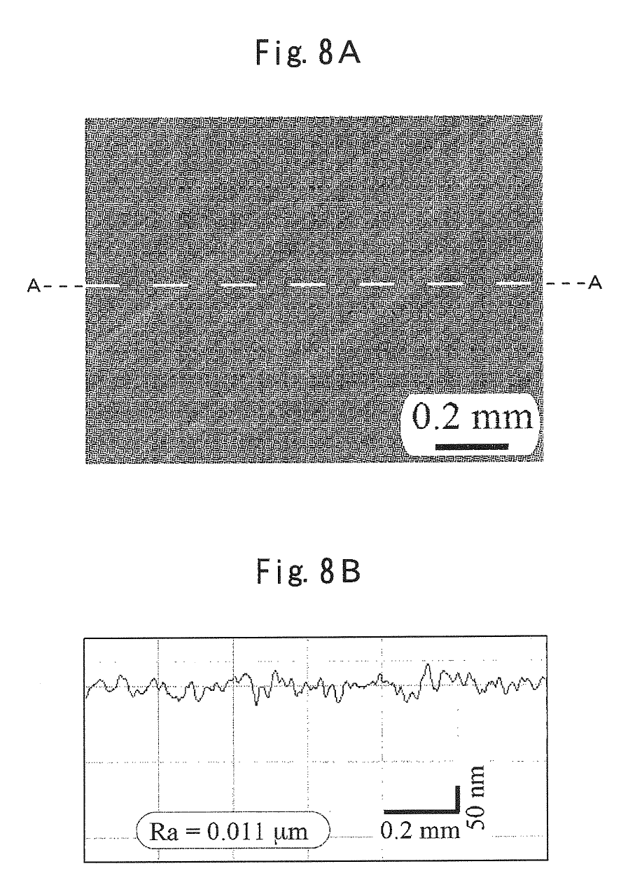

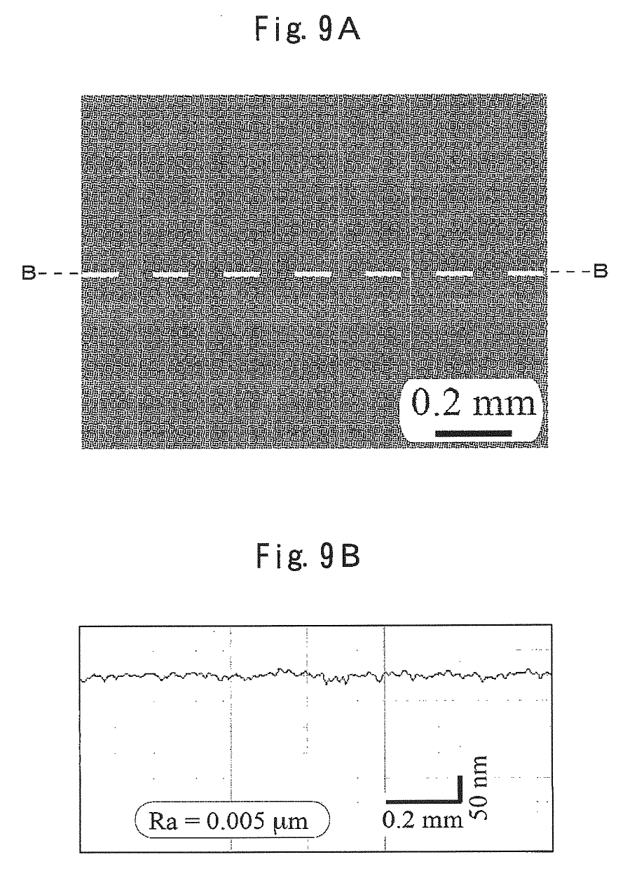

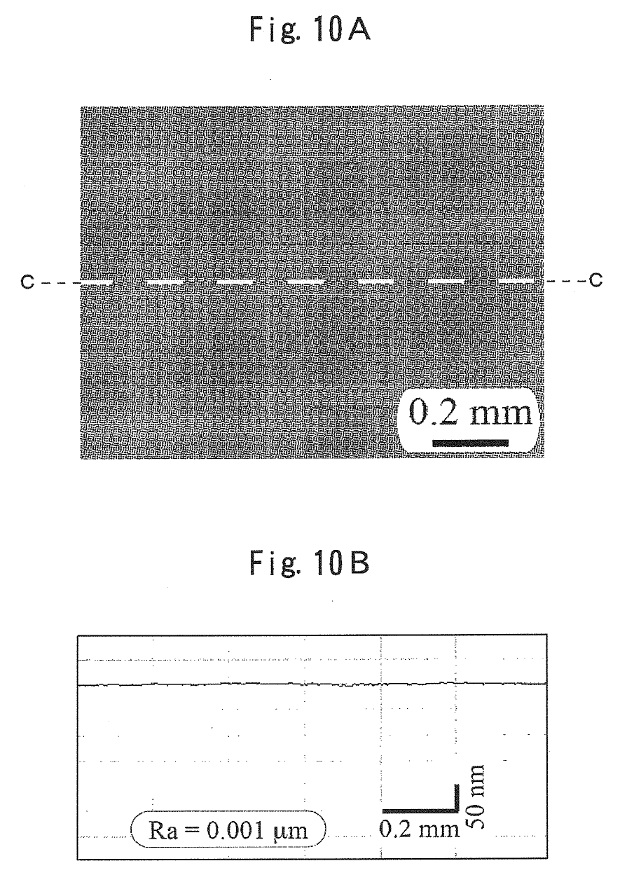

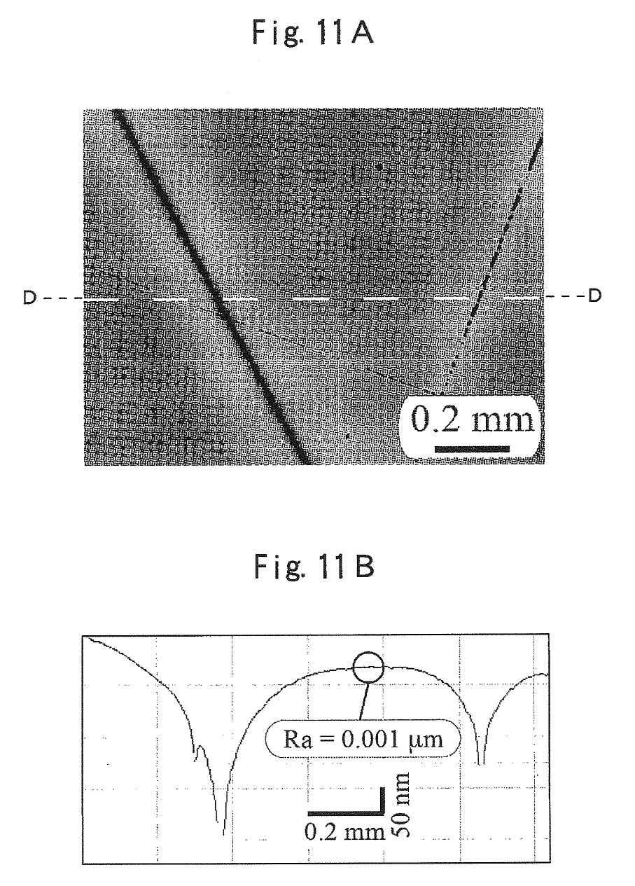

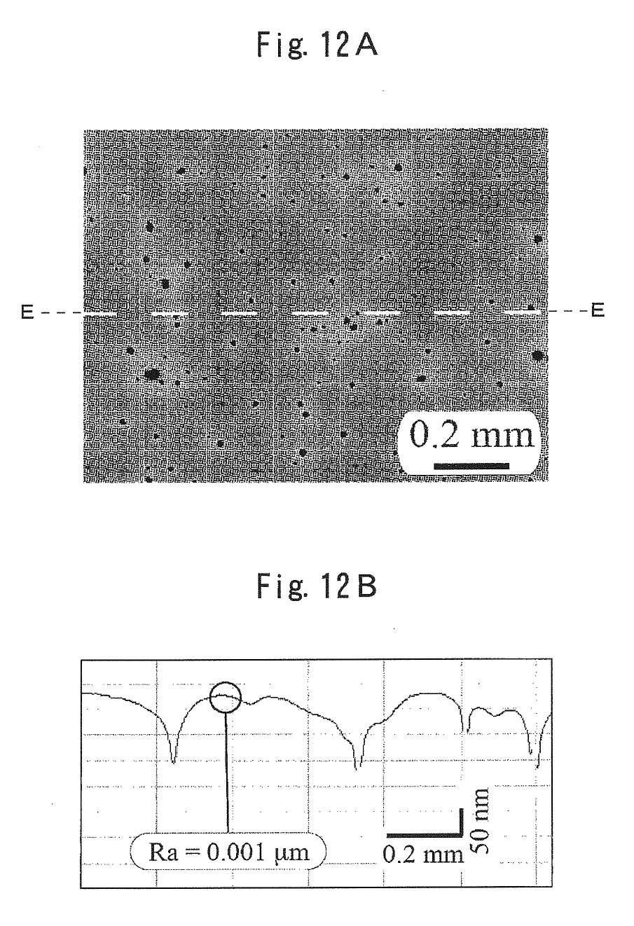

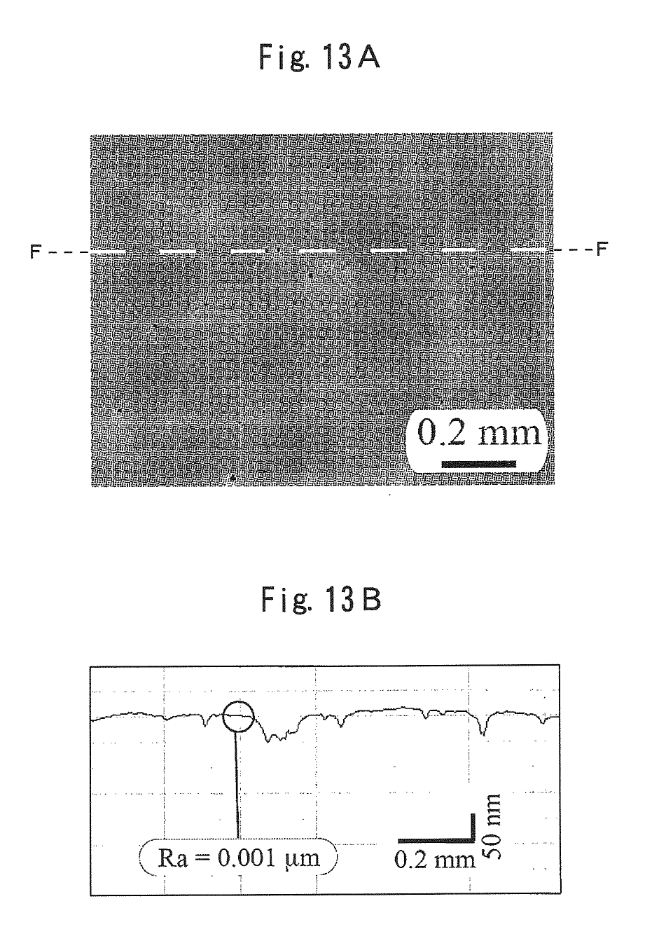

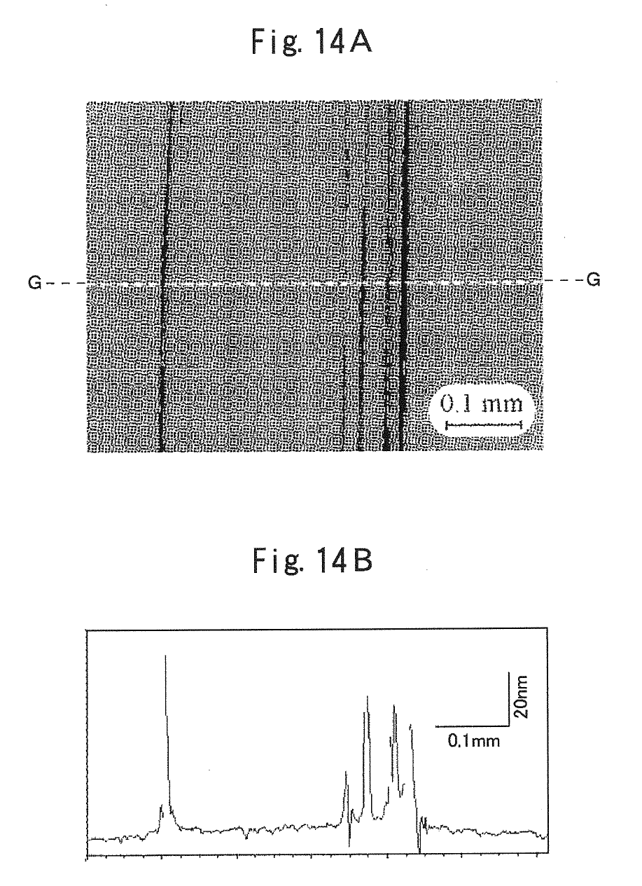

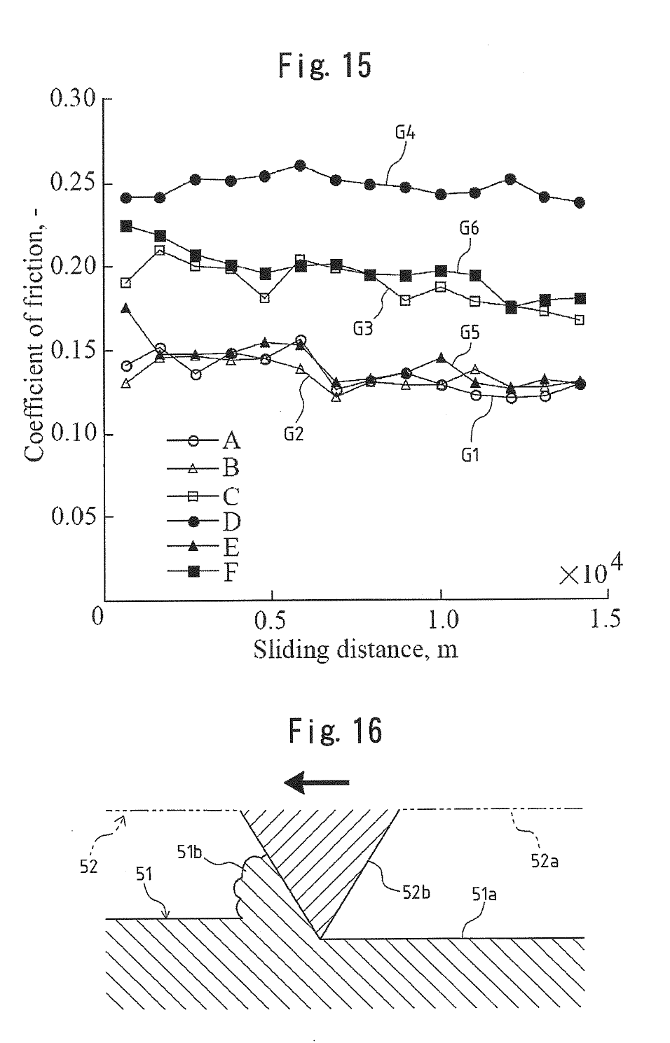

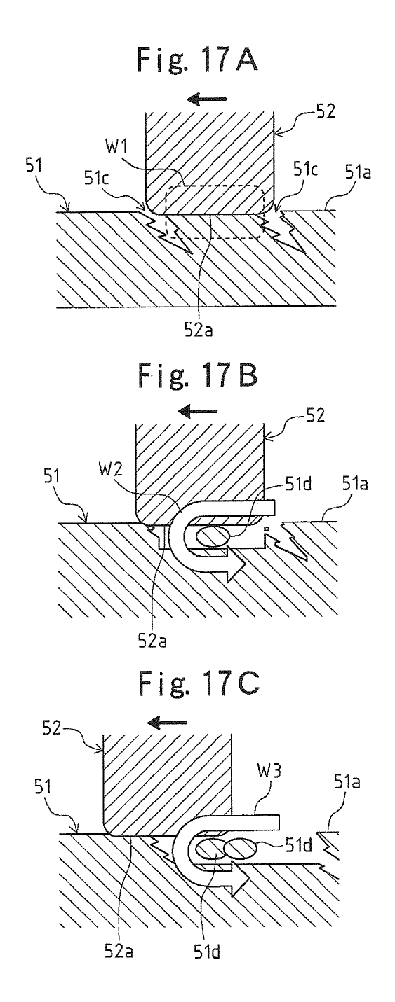

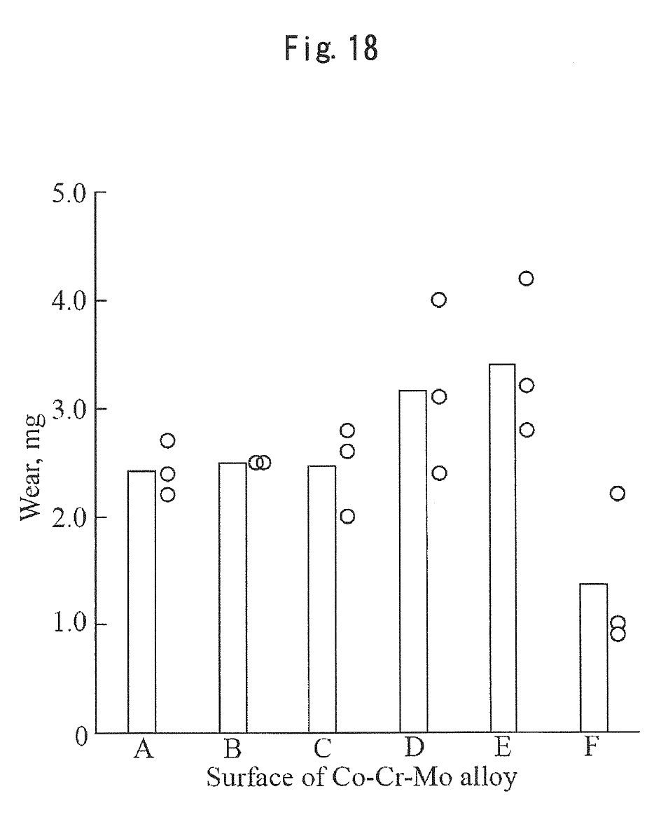

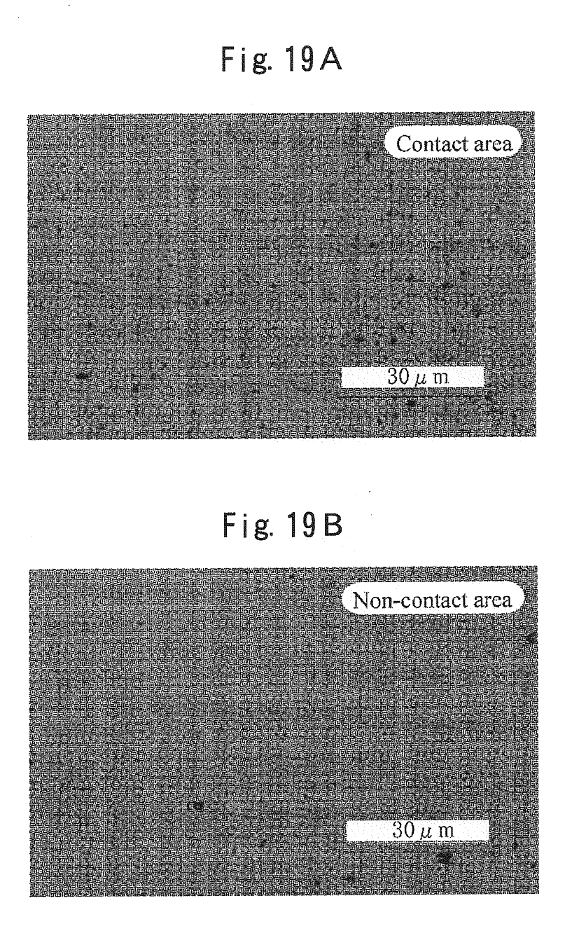

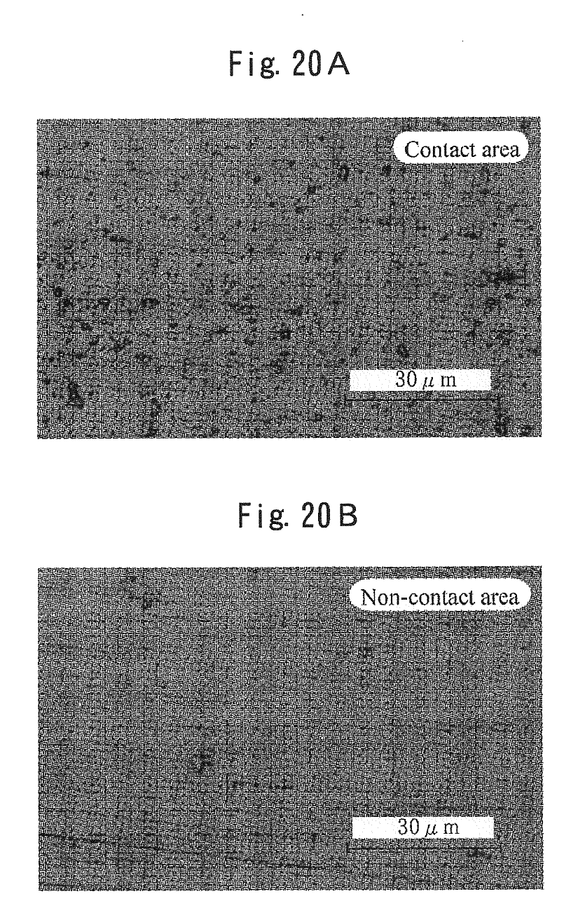

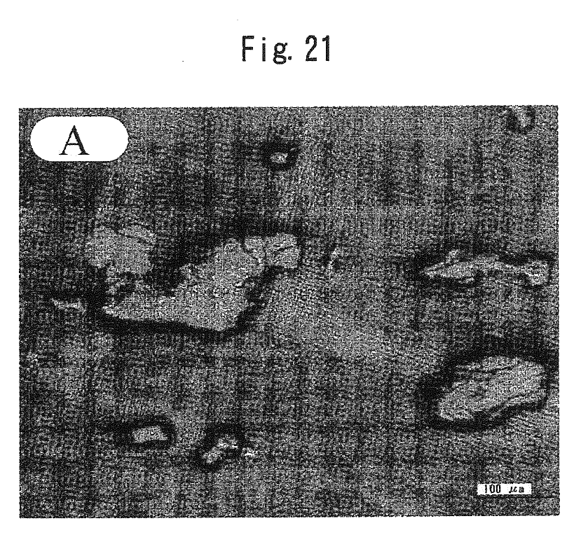

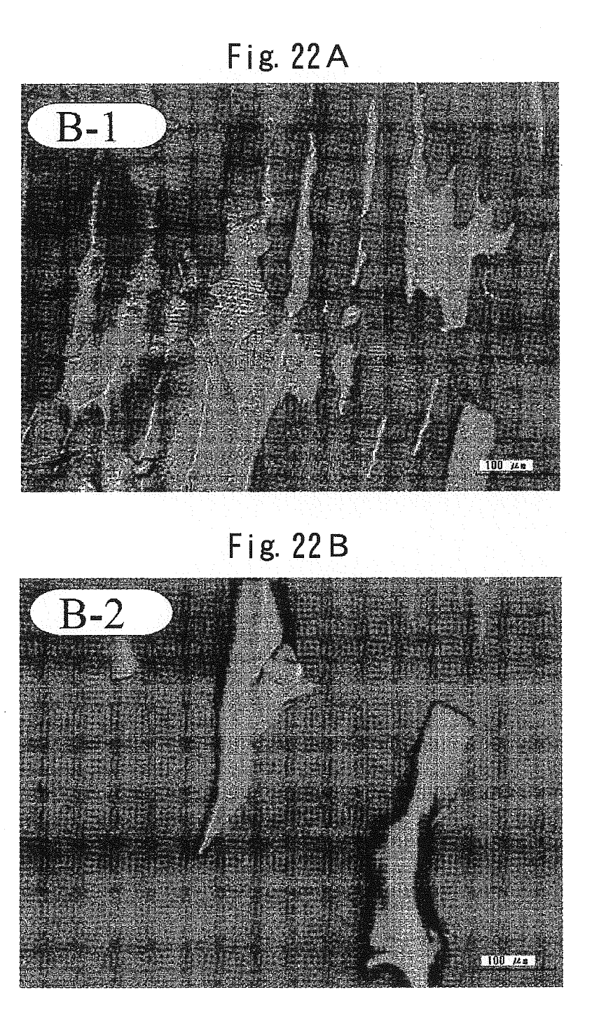

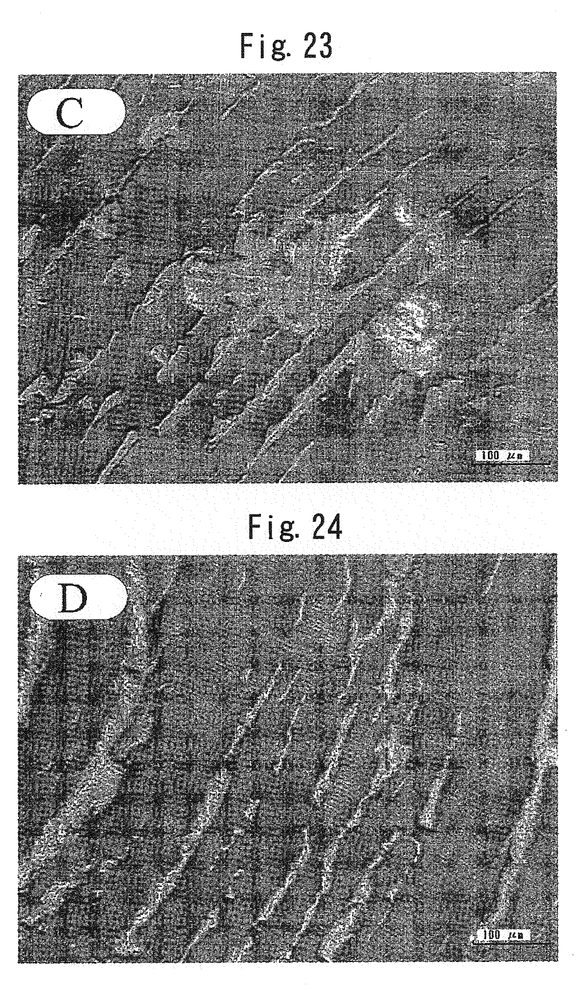

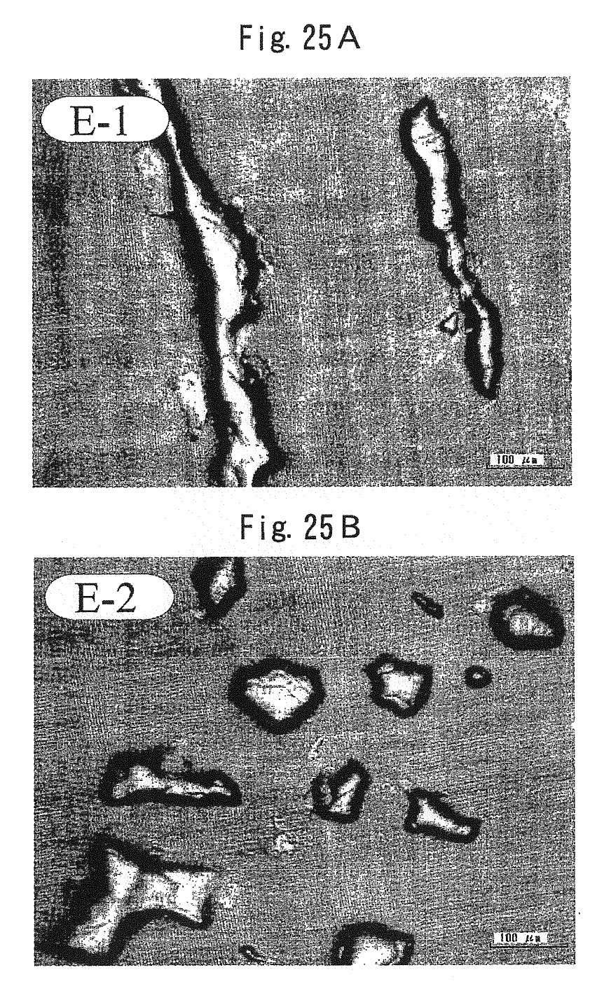

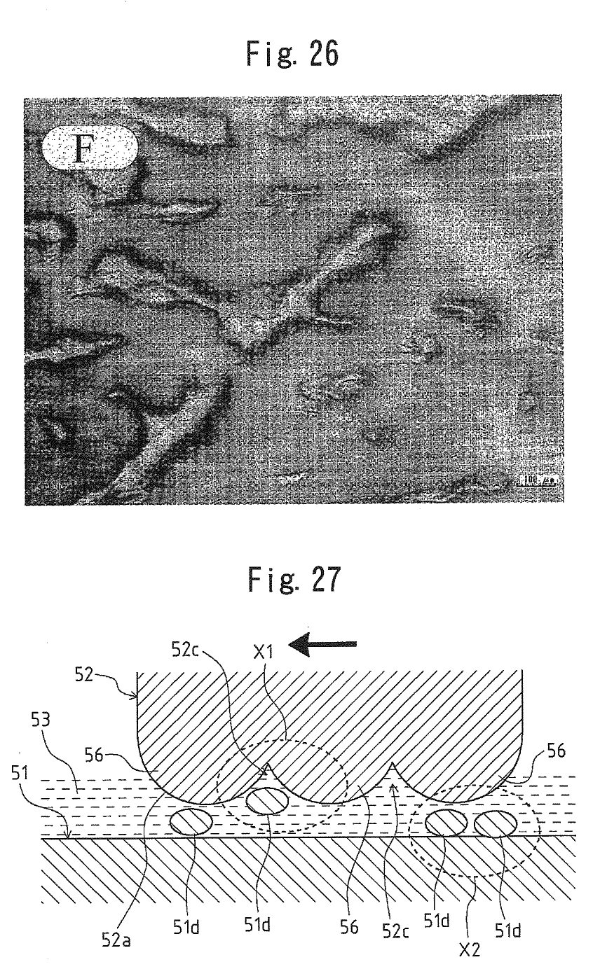

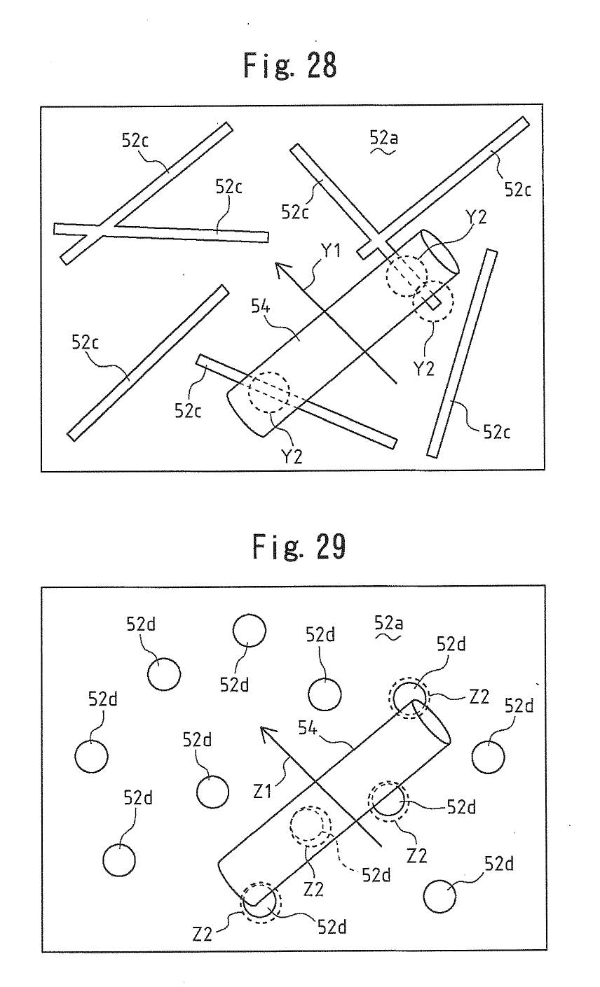

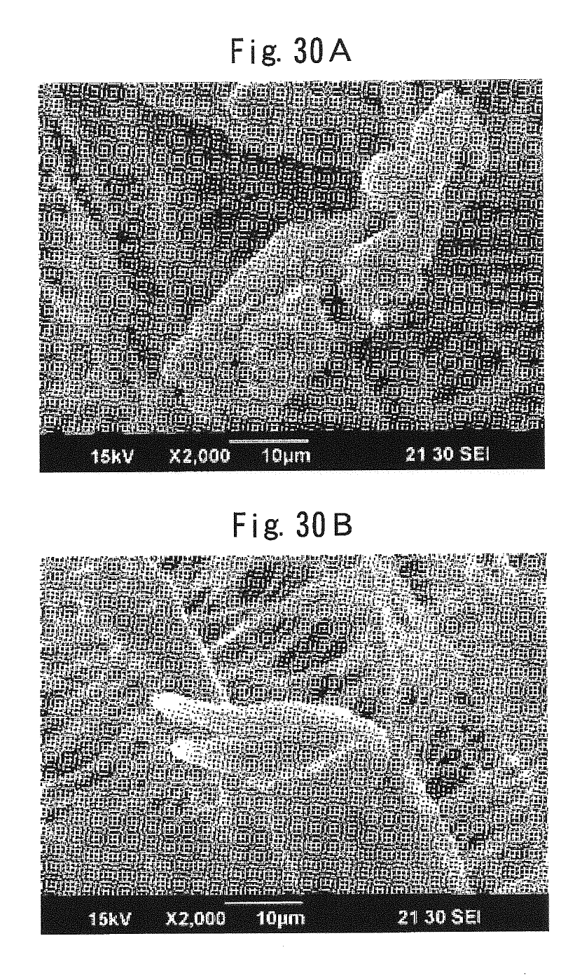

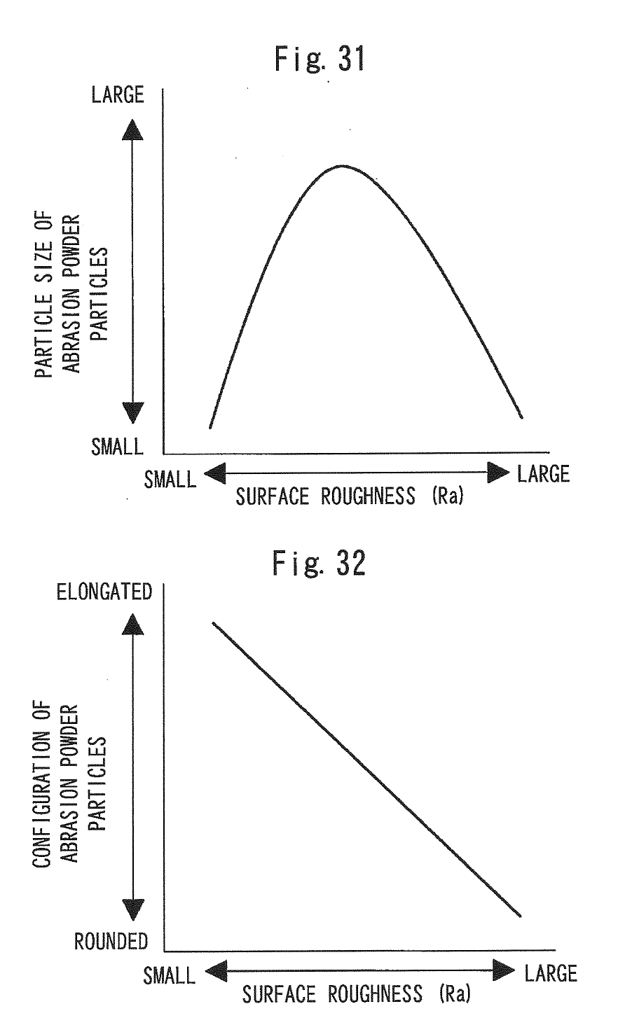

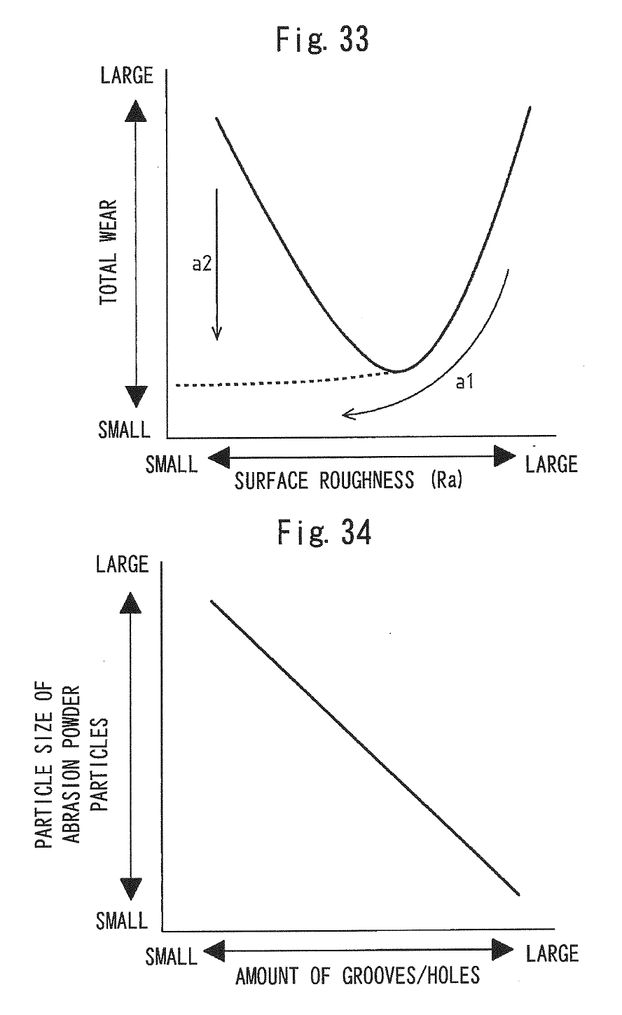

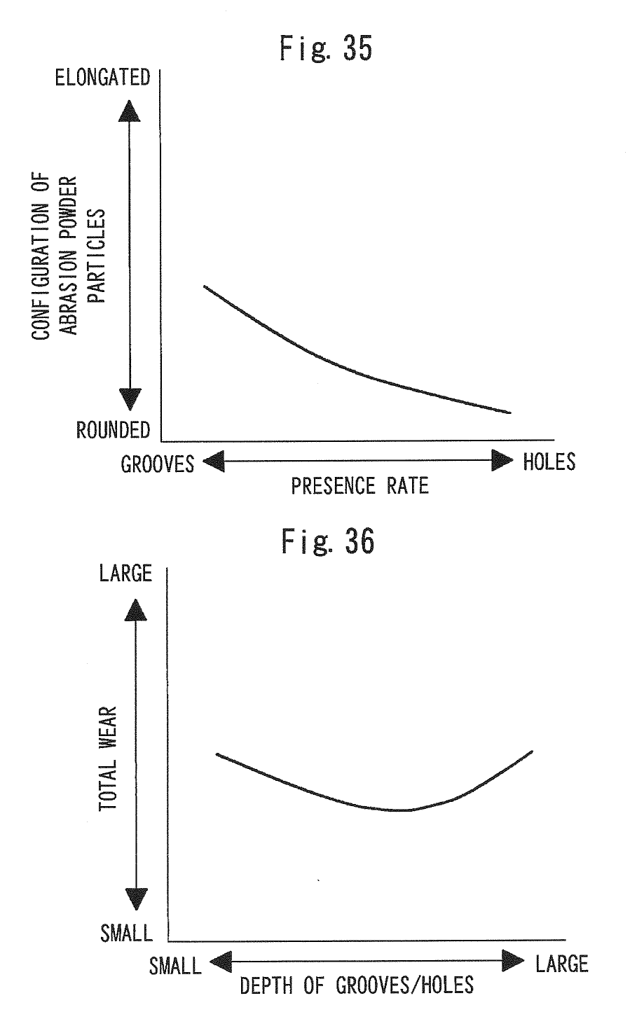

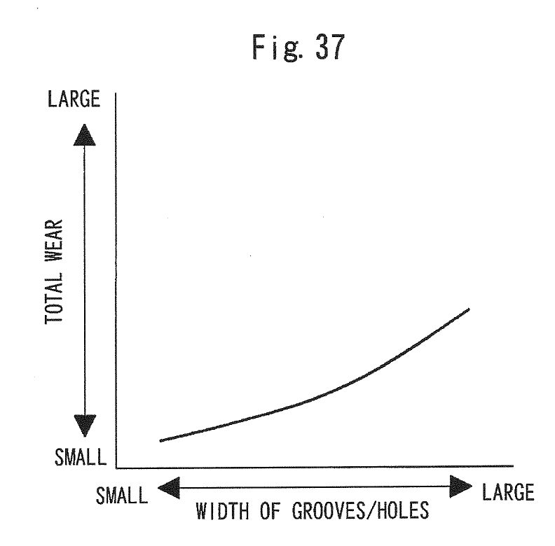

FIG. 5 is a cross-sectional view showing the friction surface structure in the artificial joint according to one mode for carrying out the present invention;

FIG. 6 is a view schematically showing a test device according to the embodiment of the present invention;

FIG. 7 is a view showing a table on contents lubrication fluids according to the embodiment of the present invention;

FIG. 8 is a view showing a surface analysis result of a first comparison example product according to the embodiment of the present invention.

FIG. 9 is a view showing a surface analysis result of a second comparison example product according to the embodiment of the present invention;

FIG. 10 is a view showing a surface analysis result of a third comparison example product according to the embodiment of the present invention;

FIG. 11 is a view showing a surface analysis result of a first embodiment product according to the embodiment of the present invention;

FIG. 12 is a view showing a surface analysis result of a second embodiment product according to the embodiment of the present invention;

FIG. 13 is a view showing a surface analysis result of a third embodiment product according to the embodiment of the present invention;

FIG. 14 is a view showing a surface analysis result of scratch flaws according to the embodiment of the present invention;

FIG. 15 is a view showing one example of the result of measurement of the transition of a coefficient of friction in an experiment according the embodiment of the present invention;

FIG. 16 is an explanatory view for explaining cutting-type wear according to the embodiment of the present invention;

FIG. 17 is an explanatory view for explaining coagulation-type wear according to the embodiment of the present invention;

FIG. 18 is a view showing one example of a measurement result of a wear weight of ultra-high molecular weight polyethylene after an experiment according to the embodiment of the present invention;

FIG. 19 is a view showing one example of a microscope photograph of a friction surface according to the embodiment of the present invention;

FIG. 20 is a view showing one example of a microscope photograph of a friction surface according to the embodiment of the present invention;

FIG. 21 is a view showing a microscope photograph of a surface of ultra-high molecular weight polyethylene corresponding to the first comparison example product according to the embodiment of the present invention;

FIG. 22 is a view showing a microscope photograph of a surface of ultra-high molecular weight polyethylene corresponding to the second comparison example product according to the embodiment of the present invention;

FIG. 23 is a view showing a microscope photograph of a surface of ultra-high molecular weight polyethylene corresponding to the third comparison example product according to the embodiment of the present invention;

FIG. 24 is a view showing a microscope photograph of a surface of ultra-high molecular weight polyethylene corresponding to the first embodiment product according to the embodiment of the present invention;

FIG. 25 is a view showing a microscope photograph of a surface of ultra-high molecular weight polyethylene corresponding to the second embodiment product according to the embodiment of the present invention;

FIG. 26 is a view showing a microscope photograph of a surface of ultra-high molecular weight polyethylene corresponding to the third embodiment product according to the embodiment of the present invention;

FIG. 27 is an explanatory view showing the manner of operation of recessed portions according to the embodiment of the present invention;

FIG. 28 is an explanatory view showing the manner of operation of recessed portions according to the embodiment of the present invention;

FIG. 29 is an explanatory view showing the manner of operation of recessed portions according to the embodiment of the present invention;

FIG. 30 is a view showing a microscope photograph as an observation example of wear particles according to the embodiment of the present invention;

FIG. 31 is a view showing the relationship between the surface roughness of a friction surface and particle sizes of wear particles according to the embodiment of the present invention;

FIG. 32 is a view showing the relationship between the surface roughness of a friction surface and the configuration of wear particles according to the embodiment of the present invention;

FIG. 33 is a view showing the relationship between the surface roughness of a friction surface and a total wear quantity according to the embodiment of the present invention;

FIG. 34 is a view showing the relationship between an amount of recessed portions and particle sizes of wear particles according to the embodiment of the present invention;

FIG. 35 is a view showing the relationship between a presence ratio of the recessed portions and the configuration of wear particles according to the embodiment of the present invention;

FIG. 36 is a view showing the relationship between a depth of the recessed portions and a total wear quantity according to the embodiment of the present invention; and

FIG. 37 is a view showing the relationship between a width of the recessed portions and the total wear quantity according to the embodiment of the present invention.

### DESCRIPTION OF THE PREFERRED EMBODIMENTS

The present invention is provided for decreasing wear on a friction surface such as a slide surface between members which constitute a joint in an artificial joint by improving a shape of the friction surface, and also for suppressing the elevation of bioactivity of macrophages generated when macrophages eat wear particles in such a manner that a wear mechanism is changed by controlling fine shapes (texture) of the friction surface thus controlling the configuration of wear particles generated from the friction surface. The embodiment of the present invention is explained hereinafter.

As shown in FIG. 1, in the friction surface structure according to this embodiment, there is provided a friction surface 1 which slides relative to the other member in a state where the friction surface 1 is in contact with the other member by way of a lubrication fluid. That is, the friction surface 1 is provided as a surface of a predetermined member and slides, in a state where the friction surface 1 is in contact with a friction surface of a counterpart formed by the other member 2 (hereinafter referred to as “counterpart friction surface”), relative to the counterpart friction surface 2 due to the relative movement between the members which are in contact with each other. Here, “a state where the friction surface 1 is in contact with the other member by way of a lubrication fluid” also includes a state where a lubrication fluid is interposed between the friction surface 1 and the other member and a slight gap exists between the friction surface 1 and the other member. The friction surface 1 includes groove-shaped recessed portions 3 and curved surface portions 4.

The recessed portion 3 is formed such that a width of the recessed portion 3 is gradually narrowed from a surface side of the friction surface 1 (an upper side in FIG. 1) to the inside of the friction surface 1 (a lower side in FIG. 1). The recessed portions 3 constitute groove portions which extend in the direction perpendicular to a plane of paper on which FIG. 1 is described. In the recessed portion 3, a size in a widthwise direction (lateral direction in FIG. 1) is gradually narrowed toward a deep side in the depth direction (a lower side in the same drawing).

In this embodiment, the recessed portion 3 is formed, as viewed in cross section in FIG. 1, into an acute-angle shape such that a widthwise size is gradually narrowed toward a bottom side (a deep side in the depth direction). Accordingly, the recessed portion 3 has a pair of inclined surface portions 3a which intersects with each other at a bottom side portion as viewed in cross section in FIG. 1.

In this embodiment, a large number of recessed portions 3 are formed on the friction surface 1 as linear groove portions in random arrangement. Accordingly, the recessed portion 3 is present on the friction surface 1 in such a manner that the recessed portion 3 intersects with other recessed portions 3 or is present independently without intersecting with other recessed portions 3. The recessed portions 3 may be formed as curved groove portions or a plurality of recessed portions 3 may be formed with predetermined directivity. Further, the recessed portions 3 may be formed into a hole shape. When the recessed portions 3 are fowled into a hole shape, a large number of dot-like recessed portions 3 are formed on a surface of the friction surface 1. Further, the recessed portions 3 may be formed in a state where the recessed portions 3 formed into a groove shape and the recessed portions 3 formed into a hole shape are present in mixture. That is, the recessed portions 3 are formed at least either as groove-shaped portions or hole-shaped portions.

The curved surface portion 4 smoothly connects a surface forming the recessed portion 3 and a surface portion of the friction surface 1. In this embodiment, as shown in FIG. 1, the surface portion of the friction surface 1 is formed of a planar portion 5 which extends along a surface shape of the friction surface 1. Accordingly, the curved surface portion 4 is formed as a convex curved surface which smoothly connects the inclined surface portion 3a and the planar portion 5 at a ridge portion 1 between the inclined surface portion 3a which forms the recessed portion 3 and the planar portion 5.

In this manner, on the friction surface 1 which includes the recessed portions 3 and the curved surface portions 4, the recessed portions 3 are formed around the planar portion 5 by way of the curved surface portions 4. That is, the planar portion 5 is formed at a portion between a plurality of recessed portions 3 by way of the curved surface portions 4. That is, the friction surface 1 includes, due to the formation of a plurality of recessed portions 3, convex portions 6 each of which is formed of the inclined surface portions 3a, the curved surface portions 4 and the planar portion 5.

Surfaces which form the respective portions of the friction surface 1 are worked such that the surfaces acquire the sufficiently smooth surface roughness (small Ra value) compared to, for example, general surface roughness of a surface of a metallic material to which ultra-precision texture working is applied (Ra value indicative of surface roughness being approximately 0.01 μm).

In this embodiment, the recessed portion 3 is formed such that a width of the recessed portion 3 is continuously narrowed toward a bottom side by having the inclined surface portions 3a. However, the recessed portion 3 may be formed such that a width of the recessed portion 3 is narrowed in a stepwise manner by forming the recessed portion 3 in a stepwise manner, for example. That is, it is sufficient that the groove-shaped or hole-shaped recessed portions 3 are formed such that a width of the recessed portion 3 is narrowed toward a bottom side continuously or in a stepwise manner.

One example of a working method for forming the friction surface 1 according to the present invention is explained. The working method according to this embodiment is preferably used when a metallic material such as a Co—Cr (cobalt-chromium)-based alloy, for example, is used as a friction material having the friction surface 1.

In the working method according to this embodiment, firstly, a step where lapping is performed is executed. Lapping is performed in such a manner that a platen for lapping and a friction material which is an object to be worked are rotated in a rubbing manner in a state where diamond abrasive grains and an abrasive agent are interposed therebetween so that the friction material is ground by minute cutting. Due to lapping, as shown in FIG. 2A, a large number of recessed portions 3 are formed on a surface of the friction material. That is, by performing lapping, on the surface of the friction material, a plurality of convex portions 6a having the inclined surface portions 3a which form the recessed portions 3 and the planar portions 5 are formed.

Next, a step where polishing (mirror finishing) is performed is executed. Polishing is performed using a platen for polishing and minute abrasive grains, and is performed as grinding higher than lapping. Due to polishing, as shown in FIG. 2B, an edge portion of a ridge portion between the inclined surface portion 3a which forms the recessed portions 3 and the planar portion 5 (see FIG. 2A) is shaved by polishing so that the curved surface portion 4 is formed. That is, by performing polishing, a plurality of concave portions 6a having the inclined surface portions 3a which form the recessed portions 3, the curved surface portions 4 and the planar portions 5 are formed on the surface of the friction material.

In this manner, in the working method according to this embodiment, the friction surface 1 having the recessed portions 3 and the curved surface portions 4 is obtained by mainly executing the steps consisting of lapping and polishing in two stages. However, the working method for forming the friction surface 1 is not particularly limited. As the working method for forming the friction surface 1, it is possible to adopt a suitable method depending on a kind of a friction material which has the friction surface 1, shapes and sizes of the recessed portions 3 and the curved surface portions 4, density of the recessed portions 3 on the friction surface 1 and the like. As the working method for forming the friction surface 1, besides the above-mentioned examples, a method which uses a forming mold and the like are considered, for example.

Further, in the friction surface structure according to this embodiment, as shown in FIG. 3, a convex portion 6 may be configured such that curved surface portions 4 which are contiguously formed with the inclined surface portions 3a and form the convex portion 6 are contiguously formed with each other. That is, in this case, the convex portion 6 does not have the planar portion 5 and has a rounded-mountain-like shape as a whole by the inclined surface portions 3a of the recessed portions 3 and the curved surface portions 4. Accordingly, in the friction surface structure shown in FIG. 3, a crest portion of the convex portion 6 forms a surface portion of the friction surface 1 and the curved surface portions 4 smoothly connect the inclined surface portions 3a which form the recessed portions 3 and the crest portion of the convex portion 6. Further, on the friction surface 1, the convex portions 6 which have the planar portion 5 (see FIG. 1) and the convex portions 6 which do not have the planar portion 5 (see FIG. 3) may be formed in mixture.

Further, it is preferable that a depth of the recessed portion 3 is of a sub micron size at largest. That is, with respect to the groove-shaped or the hole-shaped recessed portion 3, it is preferable that a groove depth or a hole depth is set to 0.1 to 1.0 μm. It is more preferable that a depth of the recessed portion 3 is set to 0.1 μm or less. As the depth of the recessed portion 3, for example, a magnitude of a size taken in the direction perpendicular to an imaginary plane (passing planar portions 5 or crest portions of a plurality of convex portion 6) along the surface portion of the friction surface 1 from the imaginary plane to a bottom portion of the recessed portion 3 (a crest portion formed of two inclined surface portions 3a as viewed in cross section shown in FIG. 1) is adopted.

Due to the friction surface 1 according to this embodiment which has the above-mentioned constitution, the stay of a lubricant fluid between the friction surfaces can be enhanced so that the wear of a friction material can be suppressed. To be more specific, a lubrication fluid which is interposed between the friction surface 1 and the counterpart side friction surface 2 can be held by the recessed portions 3 and hence, the wear of the friction materials which form the friction surface 1 and the counterpart side friction surface 2 respectively can be suppressed. Further, the friction surface 1 has the curved surface portions 4 together with the recessed portions 3 and hence, compared to the structure where recessed portions are simply formed on a planar portion, a wear quantity can be reduced. That is, in the structure where recessed portions are simply formed on a planar portion, a cutting-type wear caused by scratching action by an edge formed at a ridge portion between the planar portion and the recessed portion is liable to occur. However, according to the friction surface 1 of this embodiment, the curved surface portion 4 exists between the inclined surface portion 3a which forms the recessed portion 3 and the surface portion of the friction surface 1 and hence, the cutting-type wear can be prevented whereby a wear quantity can be decreased.

Further, according to the friction surface 1 of this embodiment, a quantity, a size and a shape of wear particles generated due to friction between the friction surface 1 and the counterpart side friction surface 2 can be controlled by adjusting a size such as a depth or a width of the recessed portion 3. That is, according to the friction surface 1 of this embodiment, the recessed portion 3 is used as a shape portion for controlling at least one of a quantity, a size and a shape of wear particles by adjusting at least one of a size, a shape and the distribution density of the recessed portions 3 on the friction surface 1.

To be more specific, for example, when a friction material which forms the friction surface 1 is a metallic material and a friction material which forms the counterpart side friction surface 2 is a resin material (polymer), due to the structure which possesses the recessed portion 3 and the curved surface portions 4, wear particles having a needle-like shape are obtained as wear particles generated on the counterpart side friction surface 2. The shape portion for controlling a size or the like of wear particles is not limited to the recessed portion 3 and the curved surface portion 4 may be used as the shape portion. In this case, a size or the like of the wear particles is controlled by adjusting a shape, a size or the like of the curved surface portion 4.

A mechanism that wear particles are formed into a needle-like shape by the friction surface 1 according to this embodiment is not sufficiently clarified. However, it is thought that a length, a diameter or the like of needle-like wear particles can be controlled by adjusting a size such as a depth or width of the recessed portion 3 or the like. The formation of the needle-like wear particles is advantageous with respect to a following point. That is, when the friction surface 1 according to this embodiment is used as a friction surface of an artificial joint, compared to a case where wear particles have a shape close to a spherical shape, the bio-reaction such as a phagocytosis of macrophages can be suppressed. That is, according to the friction surface 1 of this embodiment, by making the mechanism of wear different from the conventional friction surface which has neither the recessed portions 3 nor the curved surface portions 4, the configuration of wear particles generated from the friction surface is controlled and hence, it is possible to suppress the bioactivity of macrophages generated when macrophages eat wear particles.

The friction surface 1 according to this embodiment is applicable to a friction surface in, for example, the bearing structure which includes a rotatably mounted axial member and a bearing which supports the rotatably mounted axial member, an artificial joint which includes a pair of joint members constituting a joint by being connected to each other, or various mechanisms such as an implant material for orthopedic surgery. Accordingly, as a friction material for forming the friction surface 1 of this embodiment, various materials such as a metallic material, a resin material and ceramics are applicable. Further, the friction surface 1 of this embodiment can enhance a lubrication fluid holding property by the recessed portions 3 and the like and hence, when the friction surface 1 is applied to the bearing structure of a rotary part such as an axle in an automobile, for example, it is advantageous for holding or forming an oil film by a lubrication oil as a lubrication fluid in a state where the rotary part is rotated at a relatively low speed at the time of low speed driving, at the time of starting from a rotation stopped state or the like.

Subsequently, an artificial joint according to the present invention is explained. In this embodiment, the artificial joint is explained by taking an artificial hip joint as an example.

As shown in FIG. 4, an artificial hip joint 10 according to this embodiment includes, as a pair of joint members constituting a joint by being connected to each other, a ball 20 which constitutes an artificial caput femoris and a cup 30 which constitutes an artificial acetabular roof. The ball 20 has a spherical convex curved surface 21, and the cup 30 has an inner-spherical-shaped concave curved surface 31 corresponding to the convex curved surface 21. The ball 20 and the cup 30 are connected to each other by fitting a portion of the ball 20 which forms the convex curved surface 21 in a recessed portion of the cup 30 which forms the concave curved surface 31.

The ball 20 includes a caput femoris portion 20a which forms the convex curved surface 21 and a trunk portion 20b which projects from the caput femoris portion 20a in a trunk shape (in a shaft shape). By fixing a portion of the trunk portion 20b to a portion of a thigh bone in a human body using a bone cement or the like, the ball 20 is fixed to the thigh bone. By fixing an outer portion of the cup 30 on a side opposite to the concave curved surface 31 to a recessed portion formed on a pelvis side in a human body using a bone cement or the like, the cup 30 is fixed to a pelvis side.

The ball 20 and the cup 30 which are in a mutually connected state move relative to each other along these curved surfaces using the convex curved surface 21 and the concave curved surface 31 as friction surfaces. Accordingly, the artificial hip joint 10 functions as a joint by movably connecting the thigh bone to which the ball 20 is fixed to the pelvis side to which the cup 30 is fixed.

As shown in FIG. 5, in the artificial hip joint 10, a lubrication fluid 40 such as a body fluid is present between the convex curved surface 21 and the concave curved surface 31 which face each other in an opposed manner. That is, a lubricating fluid film is formed by the lubrication fluid 40 between the convex curved surface 21 and the concave curved surface 31. In this manner, in the artificial hip joint 10, between the ball 20 and the cup 30 which constitute the pair of joint members, the convex curved surface 21 and the concave curved surface 31 which constitute the pair of friction surfaces which slides relative to each other in a mutually contact state by way of the lubrication fluid 40 are formed. With respect to the convex curved surface 21 and the concave curved surface 31, “in a mutually contact state by way of the lubrication fluid 40” also includes a state where a lubrication fluid 40 is interposed between the convex curved surface 21 and the concave curved surface 31 and a slight gap is formed between the convex curved surface 21 and the concave curved surface 31.

Further, in the artificial hip joint 10 of this embodiment, out of the convex curved surface 21 and the concave curved surface 31 which constitute the pair of friction surfaces, the convex curved surface 21 which is formed by the ball 20 which constitutes one joint member is made of a metallic material, and the concave curved surface 31 formed by the cup 30 which constitutes the other joint member is made of a resin material.

To be more specific, in the artificial hip joint 10, as a material for forming the ball 20, for example, a metallic material having biocompatibility such as Ti (titanium), a Ti (titanium) alloy, a Co—Cr(cobalt-chromium)-based alloy, or stainless steel is used. Further, as a material for forming the cup 30, for example, a resin material having biocompatibility such as ultra-high molecular weight polyethylene, high-density polyethylene, polyacetal or an acrylic resin is used.

The artificial hip joint 10 having the above-mentioned constitution includes the above-mentioned friction surface structure in the convex curved surface 21 which constitutes a friction surface formed on a ball 20 side. Accordingly, as shown in FIG. 5, the artificial hip joint 10 includes recessed portions 23 and curved surface portions 24 on the convex curved surface 21 made of a metallic material.

The recessed portion 23 is formed such that a width of the recessed portion 23 is gradually narrowed from a surface side of the convex curved surface 21 (an upper side in FIG. 5) to the inside of the convex curved surface 21 (a lower side in the same drawing). The recessed portions 23 constitute groove portions which extend in the direction perpendicular to a plane of paper on which FIG. 5 is described. In the recessed portions 23, a size in a widthwise direction (lateral direction in FIG. 5) is gradually narrowed toward a deep side in the depth direction (a lower side in the same drawing).

In this embodiment, the recessed portions 23 are formed, as viewed in cross section in FIG. 5, in an acute-angle shape such that a widthwise size is gradually narrowed toward a bottom side (a deep side in the depth direction). Accordingly, the recessed portion 23 has a pair of inclined surface portions 23a which intersects with each other at a bottom side portion as viewed in cross section in FIG. 5.

In this embodiment, a large number of recessed portions 23 are formed on the convex curved surface 21 as linear groove portions in random arrangement. Accordingly, the recessed portions 23 are present on the convex curved surface 21 in such a manner that the recessed portion 23 intersects with other recessed portions 23 or is present independently without intersecting with other recessed portions 23. The recessed portions 23 may be formed as curved groove portions or a plurality of recessed portions 23 may be formed with predetermined directivity. Further, the recessed portions 23 may be formed into a hole shape. When the recessed portions 23 are formed into the hole shape, a large number of dot-like recessed portions 23 are formed on a surface of the convex curved surface 21. Further, the recessed portions 23 may be formed in a state where the recessed portions 23 formed into a groove shape and the recessed portions 23 formed into a hole shape are present in mixture. That is, the recessed portions 23 are formed at least either as groove-shaped portions or hole-shaped portions.

The curved surface portion 24 smoothly connects a surface forming the recessed portion 23 and a surface which forms a surface portion of the convex curved surface 21. As shown in FIG. 5, the convex curved surface 21 includes a convex portion 26 which is formed of inclined surface portions 23a of the recessed portions 23 and curved surface portions 24. The convex portion 26 is formed by contiguously connecting the curved surface portions 24a which are contiguously formed with the inclined surface portions 23a and form the convex portion 26 to each other. That is, the convex portion 26 forms a rounded-mountain-like shape as a whole by the inclined surface portions 23a of the recessed portions 23 and the curved surface portions 24. Accordingly, in this embodiment, a crest portion of the convex portion 26 forms a surface portion of the convex curved surface 21 and the curved surface portions 24 smoothly connect the inclined surface portions 23a which form the recessed portions 23 and the crest portion of the convex portion 26.

A planar portion which follows a surface shape of the convex curved surface 21 (see FIG. 1 and the planar portion 5) may be formed on the convex curved surface 21. In this case, a surface portion of the convex curved surface 21 is formed of a planar portion which follows the surface shape of the convex curved surface 21, and the curved surface portions 24 are formed as convex curved surfaces which smoothly connect the inclined surface portions 23a and the planar portion contiguously at a ridge portions between the inclined surface portions 23a which form the recessed portion 23 and the planar portion.

The recessed portions 23 and the curved surface portions 24 which the convex curved surfaces 21 have are, as described previously, formed by a working method which includes a step where lapping is performed and a step where polishing is performed. Further, surfaces which form the respective portions of the convex curved surface 21 are worked to sufficiently smooth surface roughness compared to general surface roughness of a surface of a metallic material to which ultra-precision texture working is applied, for example. With respect to the convex curved surface 21, it is preferable that a depth of the recessed portion 23 is of sub micron size at largest in the same manner as the depth of the recessed portion 3 in the friction surface structure which forms the friction surface 1 as described previously.

According to the artificial hip joint 10 of this embodiment, the stay of a lubricant fluid 40 between the convex curved surface 21 and the concave curved surface 31 can be enhanced by the recessed portions 23 so that the wear of a friction material, particularly, the wear of the concave curved surface 31 which is made of a soft resin material for a metallic material which forms the convex curved surface 21 can be effectively suppressed. Further, the convex curved surface 21 includes the curved surface portions 24 together with the recessed portions 23 and hence, compared to the structure where recessed portions are simply formed on a planar portion, a cutting-type wear generated on the concave curved surface 31 can be prevented whereby a wear quantity can be decreased. Accordingly, a lifetime which is important with respect to the artificial joint can be elongated.

Further, according to the artificial hip joint 10 of this embodiment, a quantity, a size and a shape of wear particles generated due to friction between the convex curved surface 21 and the concave curved surface 31 can be controlled by adjusting a size such as a depth or a width of the recessed portions 23 on the convex curved surface 21. That is, according to the artificial hip joint 10 of this embodiment, the recessed portion 23 is used as a shape portion for controlling at least one of a quantity, a size and a shape of wear particles generated from the friction surface by adjusting at least one of a size, a shape and the distribution density of the recessed portions 23 on the convex curved surface 21 which constitutes the friction surface.

Since the recessed portion 23 is used as the shape portion for controlling a size and the like of wear particles, a quantity, a size and a shape of wear particles can be easily controlled. Accordingly, an optimum range of surface roughness of the friction surface where a wear quantity becomes small can be selected as described previously, and a quantity of wear particles can be also controlled by adjusting a size or the like of the recessed portions 23 and hence, a wear quantity can be effectively decreased. Further, bioactivity of macrophages generated when macrophages eat wear particles can be suppressed by controlling a size of the wear particles due to the adjustment of a size or the like of the recessed portions 23.

Accordingly, in the artificial hip joint 10 of this embodiment, the wear particles are controlled to a size which can prevent macrophages from eating wear particles, for example, by controlling a size and the like of the recessed portion 23. The shape portion for controlling a size or the like of wear particles is not limited to the recessed portion 23 and the curved surface portion 24 may be used as the shape portion. In this case, a size or the like of the wear particles is controlled by adjusting a shape, a size or the like of the curved surface portion 24.

In the artificial hip joint 10 of this embodiment, wear particles having a needle-like shape are obtained as wear particles generated from the concave curved surface 31 which is made of a resin material, for example, by adjusting a size or the like of the recessed portions 23. The formation of the needle-like wear particles is advantageous in the artificial hip joint 10 with respect to a following point. That is, compared to a case where wear particles have a shape close to a spherical shape, the bio-reaction such as a phagocytosis of macrophages can be suppressed. That is, according to the artificial hip joint 10 of this embodiment, by making the mechanism of wear different from the conventional artificial hip joint which has neither the recessed portions 23 nor the curved surface portions 24 on the friction surface, the configuration of wear particles generated from the friction surface is controlled so that it is possible to suppress the bioactivity of macrophages generated when macrophages eat wear particles.

Accordingly, in the artificial hip joint 10 according to this embodiment, it is preferable that the recessed portions 23 are formed such that wear particles generated from the friction surface have a size and a shape by which the wear particles are excluded from a subject of a phagocytosis of macrophages. Here, wear particles generated from the friction surface of the artificial hip joint 10 are mainly wear particles which are generated by wear of the concave curved surface 31 made of a resin material due to a friction action of the convex curved surface 21 made of a metallic material. That is, according to the artificial hip joint 10 of this embodiment, wear particles generated from the friction surface are generated as particles having a size or a shape which prevents macrophages from eating the wear particles generated from the friction surface.

The size or the shape of wear particles generated by the friction between the convex curved surface 21 and the concave curved surface 31 can be controlled by adjusting a depth, a widthwise size or the like of the recessed portions 23 formed on the convex curved surface 21. Accordingly, in the artificial hip joint 10 of this embodiment, by adjusting a depth, a widthwise size or the like of the recessed portions 23, a size or a shape of wear particles generated in the artificial hip joint 10 is controlled to a size or a shape by which the wear particles are excluded from a subject of a phagocytosis of macrophages in a body of a patient who uses the artificial hip joint 10. A shape, a size or the like of the curved surface portions 24 on the convex curved surface 21 may be adjusted so as to control a size or the like of the wear particles.

Here, the control of a size or a shape of wear particles to a size or a shape by which the wear particles are excluded from a subject of a phagocytosis of macrophages includes following states. The first state is that a plurality of wear particles are coagulated or are integrally formed so that the wear particles are formed into large lumpy particles and hence, the size per se of the wear particles becomes large to an extent that macrophages cannot eat wear particles. Another state is that the wear particle has at least a portion thereof formed into a shape such as an elongated needle-like shape which prevents macrophages from eating wear particles. Still another state is that wear particles are formed into a cotton dust shape so that density of wear particles is lowered whereby an apparent size of the wear particles becomes a size which prevents macrophages from eating wear particles.

As a specific size or a shape of wear particles, to be more specific, for example, in a case of spherical wear particles, wear particles which have a diameter of more than 1 μm including an apparent size of wear particles can be listed as wear particles of a size or a shape by which the wear particles are excluded from a subject of a phagocytosis of macrophages. In the same manner, in a case of wear particles having a needle-like elongated shape, wear particles having a length of more than 1 μm can be named as wear particles of a size or a shape by which the wear particles are excluded from a subject of a phagocytosis of macrophages.

In this manner, by forming the recessed portions 23 such that the wear particles generated from the friction surface have a size or a shape by which the wear particles are excluded from a subject of a phagocytosis of macrophages, it is possible to effectively suppress the bioactivity of macrophages generated when macrophages eat wear particles generated from the artificial hip joint 10. Accordingly, it is possible to suppress osteolysis around the artificial hip joint 10 which becomes a cause of loosening the artificial hip joint 10. As a result, a lifetime of the artificial hip joint 10 can be prolonged, and the number of replacement operations of the artificial hip joint 10 which imposes a burden on a patient who uses the artificial hip joint 10 can be minimized.

In this embodiment, although the explanation has been made with respect to the example where the artificial joint is the artificial hip joint 10, the present invention is applicable to various artificial joints such as an artificial knee joint or an artificial elbow joint besides the artificial hip joint. Further, in the artificial joint according to the present invention, as the combination of friction materials which form a pair of friction surfaces, besides the combination of a metal material and a resin material, the combination of metallic materials, the combination of resin materials, the combination including ceramics or the like may be used. Further, it is sufficient that the friction surface structure such as the convex curved surfaces 21 which have the recessed portions 23 and the curved surface portions 24 are provided to at least one friction surface out of the pair of friction surfaces formed on the artificial joint.

Hereinafter, an embodiment of the present invention is explained. This embodiment is directed to a case where the friction surface structure according to the present invention is applied to a friction surface formed using a Co—Cr—Mo alloy in an artificial joint which is the combination of friction materials formed of the Co—Cr—Mo (cobalt chrome molybdenum) alloy which is a hard material and Ultra-High Molecular Weight Polyethylene (UHMWPE).

It has been known that surface roughness of a hard material which forms a counter part friction surface is a factor which influences a wear characteristic of UHMWPE used as a bearing material of an artificial joint. Further, it has been also known that under lubrication, the smaller the surface roughness of a hard material, the smaller the wear of UHMWPE becomes. Such results are considered reasonable because bearing surfaces of the artificial joint are not completely separated from each other due to a lubrication fluid so that a friction occurs within a range from a boundary to mixed lubrication.

On the other hand, it has been also known that the wear is lowered when a contact surface pressure between UHMWPE and a hard material is increased. This mechanism has been explained such that when the contact surface pressure between UHMWPE and the hard material is increased, a lubrication film is broken in terms of a surface roughness level of a bearing surface so that the bearing surface is brought into a non-lubrication state whereby the wear is likely to be lowered due to the formation of a transferred film of UHMWPE.

Assuming that a non-lubrication region takes a ruling state, there exists a possibility of an optimum surface roughness where the wear of UHMWPE becomes minimum and hence, it may be expected that a smooth surface does not always decrease the wear of UHMWPE. In this embodiment, ultra precision profile working is applied to a bearing surface (friction surface) formed using a Co—Cr—Mo alloy as a hard material, and the relationship between a surface profile and the wear property of UHMWPE is investigated.

FIG. 6 shows the schematic constitution of a test device according to this embodiment. As shown in FIG. 6, the test device according to this embodiment is a pin-on-disk-type wear tester, and in a state where a pin 51 made of UHMWPE (average molecular weight: 6,000,000) having a diameter of 12.0 mm is brought into contact with a disk 52 made of a Co-28Cr-6Mo alloy, these parts are made to slide relative to each other. In this embodiment, as shown in FIG. 6, the pin 51 is brought into contact with a friction surface 52a which is an upper-side surface of the disk 52 at a contact surface pressure of 1.5 MPa, and multi-directional sliding is performed by setting a range of 20.0 mm squares at a center portion of the friction surface 52a as a friction range at a speed of 12.12 mm/s until a total sliding distance becomes 15.0 km. Further, as a lubrication fluid interposed between the pin 51 and the disk 52, a pseudo lubrication fluid in table shown in FIG. 7 is used.

In this embodiment, as disks 52 which are used in an experiment, three embodiment products which the friction surface structure according to the present invention is applied and three comparison example products are prepared. As the first comparison example product, a product where a general surface state is adopted as surface texture of the disk 52 (Ra=0.011 μm) is prepared (see FIG. 8). As the second comparison example product, a product where surface roughness of a friction surface 52a is approximately ½ of the corresponding surface roughness of the first comparison example product (Ra=0.005 μm) is prepared (see FIG. 9). As the third comparison example product, a product where surface roughness of a friction surface 52a is approximately 1/10 of the corresponding surface roughness of the first comparison example product (Ra=0.001 μm) is prepared (see FIG. 10).

Further, as the first embodiment product, a product where a friction surface 52a has the substantially equal surface roughness as the third comparison example product (Ra=0.001 μm) and recessed groove treatment of a submicron level is applied to the friction surface 52a is prepared (see FIG. 11). As the second embodiment product, a product where a friction surface 52a has the substantially equal surface roughness as the first embodiment product (Ra=0.001 μm) and recessed hole treatment of a submicron level is applied to the friction surface 52a in place of the recessed groove treatment in the first embodiment product is prepared (see FIG. 12). As the third embodiment product, a product where a friction surface 52a has the substantially equal surface roughness as the first embodiment product (Ra=0.001 μm) and recessed groove treatment and recessed hole treatment of a submicron level are applied to the friction surface 52a is prepared (see FIG. 13). That is, in the third embodiment example, groove-shaped recessed portions and hole-shaped recessed portions are present on the friction surface 52a in mixture.

The following surface working is applied to the first embodiment product. Firstly, using a platen made of vinyl chloride and having a spiral-shaped groove is used as a platen, lapping which uses diamond slurry of 2 to 4 μm as abrasive grains is performed three times with a working time of 10 minutes for each lapping. Next, using commercially available polishing pad (polishing cloth 410 made by ENGIS JAPAN Corporation) as a platen, polishing which uses diamond slurry of 2 to 4 μm as abrasive grains is performed 5 times with a working time of 10 minutes for each lapping. Due to such surface working, a large number of recessed grooves of a submicron level are formed on the surface of the first embodiment product.

Surface working substantially equal to the surface working applied to the first embodiment product is applied also to the second embodiment product. However, polishing which uses diamond slurry of 2 to 4 μm as abrasive grains is performed for a total working time of 10 hours. Due to such surface working, a large number of recessed holes of a submicron level are formed on the surface of the second embodiment product. Surface working substantially equal to the surface working applied to the first embodiment product is applied also to the third embodiment product. However, polishing which uses diamond slurry of 2 to 4 μm as abrasive grains is performed for a total working time of 10 hours. Due to such surface working, a large number of recessed grooves and recessed holes of a submicron level are formed on the surface of the second embodiment product. In the explanation made hereinafter, for the sake of convenience, the respective surfaces (friction surfaces) of the first comparison example product, the second comparison example product, the third comparison example product, the first embodiment product, the second embodiment product, and the third embodiment product are made to correspond to symbols, A, B, C, D, E and F respectively.

FIG. 8 to FIG. 13 show a result obtained by analyzing surfaces of Co—Cr—Mo alloys used in the test according to the embodiments using an optical surface roughness analyzer (“NT3300” made by WYKO Corporation). FIG. 8 shows an analytical result of the first comparison example product, wherein FIG. 8A shows the analytical result of a surface (A) of the first comparison example product, and FIG. 8B shows an analytical result of a surface shape at a cross-sectional position taken along a line A-A in FIG. 8A.

FIG. 9 shows an analytical result of the second comparison example product, wherein FIG. 9A shows the analytical result of a surface (B) of the second comparison example product, and FIG. 9B shows an analytical result of a surface shape a cross-sectional position taken along a line B-B in FIG. 9A. In the same manner, FIG. 10 shows an analytical result of the third comparison example product, wherein FIG. 10A shows the analytical result of a surface (C) of the third comparison example product, and FIG. 10B shows an analytical result of a surface shape a cross-sectional position taken along a line C-C in FIG. 10A.

FIG. 11 shows an analytical result of the first embodiment product, wherein FIG. 11A shows the analytical result of a surface (D) of the first embodiment product, and FIG. 11B shows an analytical result of a surface shape a cross-sectional position taken along a line D-D in FIG. 11A. As shown in FIG. 11A, B, a large number of linear recessed grooves formed on a surface of the first embodiment product correspond to the recessed portions 3 (recessed portions 23) according to the above-mentioned embodiment. Further, as also shown in FIG. 11A, B, between the linear recessed groove and a surface portion of the first embodiment product (an upper portion in FIG. 11B), curved surface portions which correspond to the curved surface portions 4 (curved surface portions 24) according to the above-mentioned embodiments are formed.

FIG. 12 shows an analytical result of the second embodiment product, wherein FIG. 12A shows the analytical result of a surface (E) of the second embodiment product, and FIG. 12B shows an analytical result of a surface shape a cross-sectional position taken along a line E-E in FIG. 12A. As shown in FIG. 12A and FIG. 12B, a large number of dot-like recessed holes formed on a surface of the second embodiment product correspond to the recessed portions 3 (recessed portions 23) according to the above-mentioned embodiment. Further, as also shown in FIG. 12A, B, between the dot-like recessed holes and a surface portion of the second embodiment product (an upper portion in FIG. 12B), curved surface portions which correspond to the curved surface portions 4 (curved surface portions 24) according to the above-mentioned embodiments are formed.

FIG. 13 shows an analytical result of the third embodiment product, wherein FIG. 13A shows the analytical result of a surface (F) of the third embodiment product, and FIG. 13B shows an analytical result of a surface shape a cross-sectional position taken along a line F-F in FIG. 13A. As shown in FIG. 13A, B, a large number of linear recessed grooves and dot-like recessed holes formed on a surface of the third embodiment product correspond to the recessed portions 3 (recessed portions 23) according to the above-mentioned embodiment. Further, as also shown in FIG. 13A, B, between the linear recessed grooves or the dot-like recessed holes and a surface portion of the third embodiment product (an upper portion in FIG. 13B), curved surface portions which correspond to the curved surface portions 4 (curved surface portions 24) according to the above-mentioned embodiments are formed.

The recessed grooves formed on the surface of the first embodiment product, the recessed holes formed on the surface of the second embodiment product, and the recessed grooves and the recessed holes formed on the surface of the third embodiment product, largely different from scratch flaws observed in general on a bearing surface, are grooves or holes where a surface of a portion which forms a convex portion is smooth in the same manner as the second comparison example product and the recessed portion is formed of a deep groove or hole. FIG. 14 shows an analytical result of scratch flaws formed on a surface of a Co—Cr—Mo alloy. FIG. 14A shows the scratch flaws as viewed from a surface, and FIG. 14B shows an analytical result of a surface shape at a cross-sectional position taken along a line G-G in FIG. 14A. As can be understood from the comparison among FIG. 11B, FIG. 12B, FIG. 13B and FIG. 14B, although the general scratch flaws form convex portions which project upward sharply, the recessed grooves formed on the first embodiment product, the recessed holes formed on the surface of the second embodiment product and the recessed grooves and the recessed holes formed on the surface of the third embodiment form smooth convex portions having a smooth hill-like shape.

With respect to three embodiment products and three comparison example products described above, the measurement of the transition of coefficient of friction during the experiment, and the measurement of a wear weight of UHMWPE and the surface observation of a bearing surface after the experiment are performed using a pin-on-disc type wear test.

FIG. 15 shows the relationship between a sliding distance m and a coefficient of friction as a result of the measurement of the transition of coefficient of friction during the experiment. In FIG. 15, a graph G1 indicated by a white circular dot, a graph G2 indicated by a white triangular dot, a graph G3 indicated by a white square dot, a graph G4 indicated by a black circular dot, a graph G5 indicated by a black triangular dot, and a graph G6 indicated by a black square dot respectively indicate the transition of a coefficient of friction with respect to the first comparison example product (A), the second comparison example product (B), the third comparison example product (C), the first embodiment product (D), the second embodiment product (E) and the third embodiment product (F). A value of coefficient of friction in each graph shown in FIG. 15 is an average value based on the measurement carried out plural times (approximately two to three times).

From the result shown in FIG. 15, the measured value of coefficient of friction of the first comparison example product (A) and the measured value of coefficient of friction of the second comparison example product (B) are substantially equal. Further, to compare the third comparison example product (C) with the first comparison example product (A) and the second comparison example product (B), it is found that the measured coefficient of friction of the third comparison example product (C) is larger than the measured coefficients of friction of the first and second comparison example products (A) and (B). It is thought that along with the decrease of surface roughness from the first comparison example product (A) and the second comparison example product (B) to the third comparison example product (C), a true contact area is increased so that coagulation force between friction surfaces is increased. That is, when the surface roughness of the friction surface 52a becomes smaller than a certain level, a coagulation-type wear is generated so that a friction force (coefficient of friction) is increased. On the other hand, when the surface roughness of the friction surface 52a becomes larger than a certain level, cutting-type wear is generated so that a friction force (coefficient of friction) is increased.

Accordingly, when surface roughness of a product is larger (coarser) than surface roughness of the first comparison example product (A), there exists a possibility that a friction force (coefficient of friction) attributed to cutting wear is increased compared to the first comparison example product (A). On the other hand, when surface roughness of a product is smaller (smoother) than surface roughness of the third comparison example product (C), there exists a possibility that a friction force attributed to coagulation-type wear is increased compared to the third comparison example product (C). That is, the surface roughness is decreased in order of the first comparison example product (A), the second comparison example product (B) and the third comparison example product (C) and hence, a state of the friction force is changed from a state where the influence of the cutting-type wear is large and the influence of the coagulation-type wear is small to a state where the increase of the influence of the coagulation-type wear is increased along with the decrease of the influence of the cutting-type wear so that the influence of cutting-type wear is small and the influence of the coagulation-type wear is large. In this embodiment, it is estimated that the friction force is mainly attributed to the cutting-type wear in the first comparison example product (A), the influence of both the cutting-type wear and the coagulation-type wear is large to some extent in the second comparison example product (B), and the friction force is mainly attributed to the coagulation-type wear in the third comparison example product (C).

Further, from the result shown in FIG. 15, it is measured that the coefficient of friction becomes larger in order of the first embodiment product (D), the third embodiment product (F) and the second embodiment product (E) with respect to three embodiment products.

Here, the cutting-type wear and the coagulation-type wear are specifically explained. The cutting-type wear and the coagulation-type wear explained here are wears in case of three comparison example products where recessed grooves and recessed holes which are formed on the example products are not formed.

Firstly, the cutting-type wear is explained. As shown in FIG. 16, when the surface roughness is relatively large as in the case of the first comparison example product (A), for example, a sharp projecting portion 52b which projects to a side which faces a friction surface 51a of a pin 51 made of UHMWPE (hereinafter referred to as “resin-side friction surface”) is schematically expressed on a friction surface 52a of the disk 52. Accordingly, due to a friction caused by the relative movement of the disk 52 relative to the pin 51 (see an arrow in FIG. 16), the projecting portion 52b acts so that the resin-side friction surface 51a is abraded by cutting. The wear which is generated due to cutting of the resin-side friction surface 51a by the projecting portion 52b of the disk 52 becomes the cutting-type wear in this manner. According to this cutting-type wear, portions cut by the projecting portion 52b are pushed and collected by the projecting portion 52b thus forming a coagulated product 51b, and the coagulated product 51b is separated from the resin-side friction surface 51a and grows toward wear particles of the pin 51.

Next, the coagulation-type wear is explained. As shown in FIG. 17A, when the surface roughness is relatively small as in the case of the third comparison example product (C), for example, the friction surface 52a of the disk 52 is schematically expressed as a plane in comparison with the friction surface 52a shown in FIG. 16. When the surface roughness of the friction surface 52a is small in this manner, as described above, a true contact area of the friction surface 52a is increased so that a coagulation force between the friction surfaces is increased. Accordingly, due to a friction caused by the relative movement between the pin 51 and the disk 52, a contact portion of the friction surface 52a which is in contact with the resin-side friction surface 51a (see a portion indicated by a symbol W1) forms a coagulation portion, and a crack 51c attributed to a shearing force occurs between the coagulation portion of the pin 51 and a portion around the coagulation portion on the resin-side friction surface 51a.

Then, as shown in FIG. 17B, when the friction between the pin 51 and the disk 52 progresses, the coagulation portion of the pin 51 which is coagulated with the friction surface 52a is sheared, and grows to an elongated round wear particles 51d by rolling (see an arrow W2). Further, as shown in FIG. 17C, when the friction between the pin 51 and the disk 52 further progresses, some of elongated and round wear particles 51d are coagulated or formed into integral bodies thus forming lumps of wear particles 51d.

FIG. 18 shows a measurement result of wear (mg) of UHMWPE after an experiment. In FIG. 18, graphs of wears of UHMWPE respectively corresponding to the first comparison example product (A), the second comparison example product (B), the third comparison example product (C), the first embodiment product (D), the second embodiment product (E) and the third embodiment product (F) are shown in order from left. Each graph in FIG. 18 indicates average values of values obtained by measurement of three times indicated by a circular mark on a side of each graph.

From the result shown in FIG. 18, it is observed that the wear of the first comparison example product (A), the wear of the second comparison example product (B) and the wear of the third comparison example product (C) are substantially equal. However, as described above, the influence of the cutting-type wear and the coagulation type wear on these three comparison example products is changed corresponding to a change of surface roughness. Accordingly, it is understood that with respect to these three comparison example products, along with the change of the surface roughness, the mechanism of wear is changed although a total wear is not largely changed.

On the other hand, with respect to the first embodiment product (D) to which recessed groove treatment is applied and the second embodiment product (E) to which recessed hole treatment is applied, compared to three comparison example products, tendency where the wear of UHMWPE which constitutes the counter part material is increased is observed. Further, with respect to the first embodiment product (D) and the second embodiment product (E), a substantially equal wear is observed. From this result, it is estimated that the formation of the recessed grooves on the friction surface 52a and the formation of the recessed holes on the friction surface 52a can acquire the substantially same advantageous effects with respect to a total wear.

Further, it can be estimated that the wears of the first embodiment product (D) and the second embodiment product (E) become larger than wears of three comparison example products because of the following reason. In a case of the first embodiment product (D) and the second embodiment product (E), due to the convex portions (see FIG. 5, convex portions 26) which are formed together with the recessed grooves or the recessed holes, in addition to the generation of the above-mentioned cutting-type wear, UHMWPE which constitutes the pin 51 enters the recessed grooves or the like since UHMWPE is softer than a hard material which constitutes the disk 52 on which the recessed grooves or the like are formed whereby cracks occur due to a shearing force in the resin side friction surface 51a. Then, when the friction between the pin 51 and the disk 52 progresses, the cracks which occur in the resin-side friction surface 51a grow so that a part of the UHMWPE is separated from the resin-side friction surface 51a in a powdery form thus generating wear particles.

While acquiring the measurement result shown in FIG. 18, also obtained is a measurement result that when the surface roughness of the friction surface 52a is substantially equal, since a large number of recessed grooves are formed on the friction surface 52a, a lubrication fluid can be easily interposed between the friction surfaces whereby a coagulation action between the friction surfaces is alleviated thus acquiring a friction coefficient reduction effect, that is, a friction reducing effect.

FIG. 19 and FIG. 20 show observation examples (microscope photographs of a surface (friction surface 52a) of a Co—Cr—Mo alloy disk (disk 52) after an experiment carried out using a digital optical microscope (“VH-6300” made by KEYENCE CORPORATION). FIG. 19 shows the microscope photograph of a case where the friction surface 52a has a large number of recessed grooves as in the case of the first embodiment product, wherein FIG. 19A shows a surface of a contact area which is in slide contact with the pin 51, and FIG. 19B shows a surface of a non-contact area which is not in contact with the pin 51. In the same manner, FIG. 20 shows a microscope photograph of a case where recessed portions such as recessed grooves are not formed on the friction surface 52a as in the case of the first comparison example product.

Due to the comparison of (a) and (b) in FIG. 19 and FIG. 20 respectively, it is understood that transferred substances or coagulation substances of UHMWPE are present on a surface of the contact area which is in slide contact with the pin 51 as expressed by black spots. Further, as can be understood from the comparison of FIG. 19A and FIG. 20A, there is observed a tendency that the transferred substances or the coagulation substances of UHMWPE are smaller in the case where the friction surface 52a has the recessed grooves. From this inclination, it is expected that coagulation substances on a Co—Cr—Mo alloy side is decreased by the recessed groove treatment so that a lubrication state is improved.

FIG. 21 to FIG. 26 show surface observation examples (microscope photographs) of UHMWPE after an experiment carried out by a confocal laser microscope (“VK-8510” made by KEYENCE CORPORTAION). FIG. 21 is the microscope photograph of UHMWPE corresponding to the first comparison example product (A), FIG. 22 is the microscope photograph of UHMWPE corresponding to the second comparison example product (B), FIG. 23 is the microscope photograph of UHMWPE corresponding to the third comparison example product (C), FIG. 24 is the microscope photograph of UHMWPE corresponding to the first embodiment product (D), FIG. 25 is the microscope photograph of UHMWPE corresponding to the second embodiment product (E) and FIG. 26 is the microscope photograph of UHMWPE corresponding to the third embodiment product (F).

As shown in FIG. 21 to FIG. 26, with respect to the surfaces of UHMWPE corresponding to all conditions, orthogonal wear flaws which are considered to the influence exerted by multidirectional sliding are observed. This observation indicates that UHMWPE (pin 51) and the Co—Cr—Mo alloy disk (disk 52) are exposed to a boundary where UHMWPE and the Co—Cr—Mo alloy disk are not completely separated from each other by a lubrication liquid or a mixed lubrication range.

Further, with respect to the surfaces of UHMWPE corresponding to all conditions, on the surface of UHMWPE (on the wear flaws), coagulation substances which are considered to grow to wear particles are observed. As shown in FIG. 21, with respect to UHMWPE corresponding to the first comparison example product (A), coagulation substances present on the surface of the UHMWPE (hereinafter referred to as “surface coagulation substances”) exhibits high rate of cutting-type wear and hence, the coagulation substances are grown into a relatively round shape. On the other hand, as shown in FIG. 22, with respect to UHMWPE corresponding to the second comparison example product (B), surface coagulation substances are relatively largely influenced by both the cutting-type wear and the coagulation type wear and hence, the surface coagulation substances which are grown into an elongated shape (needle shape) (see FIG. 22A) and the surface coagulation substances which are grown into a relatively round shape (see FIG. 22B) are observed in mixture. FIG. 22A, B are microscope photographs of different places on a surface of the same UHMWPE corresponding to the second comparison example product (B). Further, as shown in FIG. 23, with respect to UHMWPE corresponding to the third comparison example product (C), the surface coagulation substances exhibit a high rate of coagulation wear and hence, the surface coagulation material is grown to an elongated shape (needle shape).

Further, as shown in FIG. 23 and FIG. 24, to compare the third comparison example product (C) and the first embodiment product (D), a lump size of surface coagulation substances in the first embodiment product (D) is smaller than a lump size of the surface coagulation substances in the third comparison example (C). It is thought that this difference in lump size is brought about by an action of the recessed grooves which the friction surface 52a has in the first embodiment product (D).

To be more specific, as shown in FIG. 27, due to the presence of the recessed grooves 52c as recessed portions on the friction surface 52a, the wear particles 51d enter the recessed grooves 52c (see parts indicated by symbol X1). Accordingly, chances that the wear particles 51d are coagulated or are formed into integral bodies are decreased and hence, the surface coagulation substances are made relatively small. Further, also due to the presence of the recessed grooves 52c on the friction surface 52a, a lubrication fluid 53 can be easily interposed between the friction surfaces so that the surface coagulation substances are hardly coagulated or are formed into integral bodies (see portions indicated by a symbol X2) whereby the surface coagulation substances become relatively small.

There is a possibility that linear coagulation substances which are found in UHMWPE corresponding to the third comparison example product (C) and UHMWPE corresponding to the first embodiment product (D) are generated due to the increase of a coagulation action which occurs when the surface roughness of the portion of a Co—Cr—Mo alloy which comes into direct contact with UHMWPE is small. As can be understood from FIG. 23 and FIG. 24, large lumps which are observed in UHMWPE corresponding to the third comparison example product (C) are not observed in UHMWPE corresponding to the first embodiment product (D). In this respect, as reasons for such observation, it is thought that, in a case of UHMWPE corresponding to the first embodiment product (D), a lubrication fluid is likely to be interposed due to the presence of a large number of recessed grooves or there exists a possibility that, as a result that wear particles enter the recessed groove portions, relatively large lumps of coagulation substances fall from the friction surface before the growth of the transferred substances. It is considered that this result is one of factors which increases the wear of UHMWPE corresponding to the first embodiment product (D) compared to UHMWPE corresponding to the third comparison example product (C) (see FIG. 18).

On the other hand, the recessed grooves corresponding to the recessed grooves of the first embodiment product (D) are not formed in the third comparison example product (C) and hence, portions which do not come into contact with UHMWPE is small in number thus giving rise to a large possibility that the coagulation substances of UHMWPE stay between the friction surfaces. As a result, it is thought that the coagulation substances grow by coagulating with each other. Further, with respect to the third comparison example product (C), it is thought that the discharge of the coagulation substances to the outside of the friction surface is suppressed and hence, the suppression of wear at the same level as the first comparison example product (A) occurs (see FIG. 18). From the result shown in FIG. 21 to FIG. 26, it is found that a surface profile of a Co—Cr—Mo alloy influences the configuration of wear particles of UHMWPE which are considered to influence the degree of expression of a bio-reaction.

As shown in FIG. 24 and FIG. 25, to compare the first embodiment product (D) having the recessed grooves on the friction surface 52a and the second embodiment product (E) having the recessed holes on the friction surface 52a, surface coagulation substances in the second embodiment product (E) are more likely to become rounded than surface coagulation substances in the first embodiment product (D). It is thought that such difference in the configuration of the surface coagulation substances is derived from the difference in action between the recessed grooves and the recessed holes which are present on the friction surface 52a. FIG. 25A, B are microscope photographs of different places on a surface of the same UHMWPE corresponding to the second embodiment product (E).

Here, the explanation is made with respect to the relevancy between the recessed grooves and the recessed holes and the configuration of the surface coagulation substances. As shown in FIG. 28, when a large number of recessed grooves 52c are present on a friction surface 52a, in a process where a surface coagulation substance 54 grows while rolling due to a friction between a pin 51 and a disc 52 (see an arrow Y1), a distance that the surface coagulation substance 54 rolls become relatively long. This is because, as shown in FIG. 28, when a large number of recessed grooves 52c are present on the friction surface 52a, due to the influence of the distribution density of the recessed grooves 52c, with respect to the surface coagulation substance 54 which grows while rolling, portions of the surface coagulation substance 54 having a possibility of being cut when pushed to the recessed grooves 52c (see portions indicated by symbol Y2) are relatively small in number. Accordingly, in the first embodiment product (D) which has a large number of recessed grooves 52c on the friction surface 52a, the surface coagulation substance 54 is likely to be elongated by rolling a relatively long distance.

On the other hand, as shown in FIG. 29, when a large number of recessed holes 52d are present on a friction surface 52a, in a process where a surface coagulation substance 54 grows while rolling due to a friction between a pin 51 and a disc 52 (see an arrow Z1), a distance that the surface coagulation substance 54 rolls become relatively short. This is because, as shown in FIG. 29, when a large number of recessed holes 52d are present on the friction surface 52a, due to the influence of the distribution density of the recessed holes 52d, with respect to the surface coagulation substances 54 which grow while rolling, portions of the surface coagulation substances 54 having a possibility of being cut when pushed to the recessed holes 52d (see portions indicated by symbol Z2) are relatively large in number. Accordingly, in the second embodiment product (E) which has a large number of recessed holes 52d on the friction surface 52a, the surface coagulation substance 54 becomes hardly elongated and is likely to be rounded by rolling a relatively short distance.

FIG. 30 shows an observation example of wear particles generated on a surface of the third embodiment product. FIG. 30A, B respectively show observation examples (microscope photographs) of a surface (friction surface 2a) of a Co—Cr—Mo alloy disc (disc 52a) after an experiment taken by an electronic microscope (SEM) (“JSM-6390LV” made by Nihon Electronic Co., Ltd.).

In the respective photographs shown in FIG. 30A, B, a lump which is present at the approximately center is wear particles. To be more specific, in the photograph shown in FIG. 30A, the wear particles having an elongated shape extending in the longitudinal direction which is the direction toward a left lower side from a right upper side is shown. Further, in the photograph shown in FIG. 30B, wear particles having a bifurcated shape at one end side (a left side in the drawing) are shown.

All wear particles shown in the respective photographs shown in FIG. 30A, B have a size of approximately several tens μm and hence, it is safe to say that the wear particles have a sufficient size or a sufficient shape by which the wear particles are excluded from a subject of a phagocytosis of macrophages. In this manner, by applying the friction surface structure according to the present invention to the third embodiment product, the generation of wear particles having a size or a shape by which the wear particles are excluded from a subject of a phagocytosis of macrophages is acquired as one result of this embodiment.

Based on the understanding of this embodiment, the relationship between the roughness (Ra value) of a direct contact portion of the friction surface 52a of the disc 52 with respect to the resin-side friction surface 51a and the friction property of the pin 51 (UHMWPE) is explained. Here, in a case of the friction surface 52a having the recessed grooves 52c and the recessed holes 52d as in the case of three embodiment product, the direct contact portions of the friction surface 52a are portions of convex portions 56 which are formed together with the recessed grooves 52c and the recessed holes 52d.

FIG. 31 is a graph showing the relationship between the surface roughness (Ra) of the friction surface 52a and a particle size of the wear particles. As shown in FIG. 31, the particle size of the wear particles becomes maximum when the surface roughness (Ra) takes a certain value, and using the value of the surface roughness (Ra) where the particle size of the wear particles becomes maximum as a boundary, there is a tendency that the particle size of the wear particles becomes gradually smaller along with a change in the value of the surface roughness (Ra) irrespective of whether the value of the surface roughness (Ra) is changed in an increasing direction or in a decreasing direction. In this embodiment, it is estimated that the value of the surface roughness (Ra) where the particle size of the wear particles becomes maximum is present between the surface roughness (Ra=0.011 μm) of the first comparison example product (A) and the surface roughness (Ra=0.001 μm) of the third comparison example product (C).

FIG. 32 is a graph showing the relationship between the surface roughness (Ra) of the friction surface 52a and the configuration of the wear particles. As shown in FIG. 32, the larger the surface roughness (Ra), the more rounded the configuration of the wear particles becomes, while the smaller the surface roughness (Ra), the more elongated the configuration of the wear particles becomes. In this embodiment, this can be understood from shapes of surface coagulation substances on UHMWPE which correspond to the first comparison product (A), the second comparison product (B) and the third comparison product (C) where the surface roughness (Ra) is increased in this order (see FIG. 21 to FIG. 23).

Based on the respective relationships among the surface roughness (Ra) of the friction surface, the particle size of the wear particles and the configuration of the wear particles, the surface roughness (Ra) of the friction surface on which the recessed grooves or the recessed holes are formed is adjusted to a proper value such that the wear particles generated from the friction surface have a particle size or the configuration which suppresses the bioactivity of macrophages. In other words, the bioactivity of macrophages can be suppressed by adjusting the surface roughness (Ra) of the friction surface on which the recessed grooves or the recessed holes are formed.

FIG. 33 is a graph showing the relationship between the surface roughness (Ra) of the friction surface 52a and the total wear. A graph indicated by a solid line in FIG. 33 is a graph showing a case where the recessed grooves and the recessed holes are not present on the friction surface 52a as in the case of three comparison example products. As can be understood from the graph indicated by the solid line, the total wear becomes minimum when the surface roughness (Ra) takes a certain value, and using the value of the surface roughness (Ra) where the total wear becomes minimum as a boundary, the total wear has a tendency where the total wear is gradually increased along with a change in the value of the surface roughness (Ra) irrespective of whether the value of the surface roughness (Ra) is changed in an increasing direction or in a decreasing direction.

This is based on that when the surface roughness (Ra) of the friction surface is increased, the cutting-type wear becomes dominant so that the wear is increased, while when the surface roughness (Ra) of the friction surface is decreased, the coagulation-type wear becomes dominant so that the wear is increased. That is, in a friction surface environment close to a dry state, it is not always the case that the smoother a surface of the friction surface (the smaller the surface roughness (Ra)), the smaller the wear becomes, and a range of an optimum value exists with respect to the surface roughness (Ra) of the friction surface from a viewpoint of decreasing the wear. In this embodiment, it is estimated that a value of the surface roughness (Ra) where the total wear become minimum is present between the surface roughness (Ra=0.011 μm) of the first comparison example product (A) and the surface roughness (Ra=0.001 μm) of the third comparison example product (C).

Further, in FIG. 33, a graph partially indicated by a broken line is a graph showing a case where recessed grooves and/or recessed holes are present on the friction surface 52a as in the case of three embodiment products. As can be understood from the graph partially indicated by a broken line, due to the presence of the recessed grooves and/or recessed holes on the friction surface 52a, the smaller the surface roughness (Ra), the smaller the total wear becomes (see an arrow a1). This is attributed to a phenomenon that a lubrication fluid is likely to be interposed between the friction surfaces due to the recessed grooves and/or recessed holes so that a gap between the friction surfaces is brought into an environment close to a wet state whereby a coagulation action between the friction surfaces is alleviated. When the gap between the friction surfaces is brought into an environment close to a wet state, the smaller the surface roughness (Ra), the smaller the wear becomes. That is, due to the formation of the recessed grooves and/or recessed holes on the friction surface 52a, the smaller the surface roughness (Ra), the more the coagulation-type wear which exerts large influence is suppressed and hence, the total wear within a range where the surface roughness (Ra) is relatively small is decreased (see an arrow a2).

Subsequently, the relationship between the recessed portions (recessed grooves and/or recessed holes) formed on the friction surface 52a of the disk 52 and the friction property of the pin 51 (UHMWPE) is explained.

FIG. 34 is a graph showing the relationship between an amount (quality per unit area, distribution density) of recessed grooves and/or recessed holes formed on the friction surface 52a and a particle size of wear particles. As shown in FIG. 34, there is a tendency that the larger an amount of the recessed grooves and/or recessed holes, the smaller the particle size of the wear particles becomes, and the smaller an amount of recessed grooves and/or recessed holes, the smaller the particle size of the wear particles becomes. This is based on a phenomenon that a surface coagulation substance which grows while rolling between the above-mentioned friction surfaces is pushed to the recessed grooves and/or recessed holes so that the surface coagulation substance is cut. That is, the larger an amount of recessed grooves and/or recessed holes on the friction surface 52a, the larger the number of portions where the surface coagulation product is cut becomes so that a particle size of the wear particles becomes small. To the contrary, the smaller an amount of recessed grooves and/or recessed holes on the friction surface 52a, the smaller the number of portions where the surface coagulation product is cut becomes so that a particle size of the wear particles becomes large.

FIG. 35 is a graph showing the relationship between a presence rate of recessed grooves and recessed holes formed on the friction surface 52a and the configuration of wear particles. Here, the presence rate of the recessed grooves and/or recessed holes is a relative rate between the recessed grooves and/or recessed holes in a state where the recessed grooves and the recessed holes are present on the friction surface 52a in mixture. As shown in FIG. 35, a wear powder configuration has a tendency that the larger the presence rate of the recessed holes, the more rounded the configuration of the wear particles becomes, while the larger the presence rate of the recessed grooves, the more elongated the configuration of the wear particles becomes. This tendency is based on the fact that, as described previously, the recessed holes include the large number of portions with a possibility of cutting the surface coagulation substances compared to the recessed grooves.

FIG. 36 is a graph showing the relationship between a depth of recessed grooves and/or recessed holes formed on the friction surface 52a and the total wear. As shown in FIG. 36, there is a tendency that the total wear becomes minimum when the depth of the recessed grooves and/or recessed holes takes a certain value, and using the value of the depth of the recessed grooves and/or recessed holes where the total wear becomes minimum as a boundary, the total wear is gradually increased along with a change in the value of the depth of the recessed grooves and/or recessed holes irrespective of a change of the depth of the recessed grooves and/or recessed holes in an increasing direction or in a decreasing direction. This tendency is based on a fact that when the depth of the recessed grooves and/or recessed holes is increased, the cutting-type wear becomes dominant so that the wear is increased, while when the depth of the recessed grooves and/or recessed holes is decreased, the coagulation-type wear becomes dominant so that the wear is increased.

FIG. 37 is a graph showing the relationship between a width of recessed grooves and/or recessed holes formed in the friction surface 52a and the total wear. Here, the width of recessed grooves and/or recessed holes corresponds to an opening area of the recessed grooves and/or recessed holes formed as recessed portions on the friction surface. As shown in FIG. 37, there is a tendency that the larger the width of the recessed grooves and/or recessed holes, the larger the total wear becomes, while the smaller the width of the recessed grooves and/or recessed holes, the smaller the total wear becomes. This tendency is based on a fact that the larger the width of the recessed grooves and/or recessed holes, the larger both the influence of cutting-type wear and the influence of coagulation-type wear become.

As described above, there exists the relevancy between the recessed grooves and/or recessed holes formed on the friction surface and wear properties of UHMWPE. Based on such relevancy, the recessed portions formed as the recessed grooves and/or recessed holes are used as shape portions for controlling at least one of a quantity, a size and a shape (configuration) of wear particles generated from the friction surface by adjusting at least one of a size, a shape and the distribution density of the recessed portions on the friction surface. Further, a size or the like of the recessed grooves and/or recessed holes is preferably adjusted to a size or a shape by which wear particles generated from the friction surface are excluded from a subject of a phagocytosis of macrophages.

As described above, in this embodiment, to suppress the wear of UHMWPE and to acquire guide lines for studying the optimum surface treatment method of a bearing surface on a hard material side for suppressing bioactivity of macrophages brought about by wear particles, the relationship between surface profiles of UHMWPE and the Co—Cr—Mo alloy and the wear of UHMWPE is investigated. From this embodiment, it is found that the simple decrease of surface roughness does not always bring about the suppression of wear, and groove working or hole working at a submicron level relating to the friction surface structure of the present invention are effective.

As a future prospect, it is necessary to propose surface profile working where groove working or hole working is performed at a depth which does not influence cutting-type wear so that a lubrication fluid can be easily introduced between the friction surfaces thus suppressing coagulation-type wear and, at the same time, suppressing bioactivity of macrophages brought about by wear particles. Further, by providing a surface profile which does not influence the surface roughness of the bearing surface while improving a lubrication state, it is considered possible to accelerate a configuration control of wear particles of UHMWPE and the suppression of total wear.

The artificial joint according to the present invention is based on a viewpoint such as a change of a working method of a friction surface on a hard material side of an artificial joint which is already clinically applied, to be more specific, the application of surface texturing of the friction surface and hence, the safety is guaranteed, and the understanding of the artificial joint according to the present invention by an industrial field which handles artificial joints is facilitated, and the entrance of the artificial joint according to the present invention in such an industrial field is also facilitated. Conventionally, a metallic material such as a titanium alloy, a cobalt-chromium-based alloy or stainless steel has been popularly used as a hard material of an artificial joint, for example. According to the present invention, while maintaining excellent properties such as favorable workability, high toughness or high ductility which these metal materials possess, it is possible to realize the completion of a lubrication system which can suppress bioactivity of macrophages brought about by wear particles generated from the friction surface while decreasing total wear or maintaining the total wear in a current state.

What is claimed is:

1. An artificial joint comprising:
a pair of joint members which constitutes a joint; and
a pair of friction surfaces which slides relative to each other, being formed between the pair of joint members in a state where the friction surfaces are brought into contact with each other by way of a lubrication fluid, wherein
at least one friction surface out of the pair of friction surfaces comprises:
at least either one of groove-shaped recessed portions and hole-shaped recessed portions having a width gradually narrowed toward the inside of the friction surface from a surface side of the friction surface; and
curved surface portions which smoothly connect surfaces which form the recessed portions and surfaces which form a surface portion of the friction surface.

2. The artificial joint according to claim 1, wherein a depth of the recessed portion is of a sub micron size at largest.

3. The artificial joint according to claim 1, wherein out of the pair of friction surfaces, the friction surface which is formed by one joint member is made of a metallic material, and the friction surface which is formed by the other joint member is made of a resin material, and the recessed portions and the curved surface portions are formed on the friction surface made of the metallic material.

4. The artificial joint according to claim 1, wherein the recessed portion is used as a shape portion for controlling at least one of a quantity, a size and a shape of wear particles generated from the friction surface by adjusting at least one of a size, a shape and the distribution density of the recessed portions on the friction surface.

5. The artificial joint according to claim 1, wherein the recessed portions are formed such that wear particles generated from the friction surface have a size or a shape by which the wear particles generated from the friction surface are excluded from a subject of a phagocytosis of macrophages.

6. An artificial bone joint, for an animal body, comprising:
a first joint member having a first friction surface;
a second joint member having a second friction surface;
said first joint member and said second joint member operably engaging as said artificial bone joint whereby said first friction surface is proximate to said second friction surface; and
a lubrication fluid filling between said first friction surface and said second friction surface;
wherein said first friction surface and said second friction surface slidely face each other with a space thereby forming said artificial joint; and at least one portion of said first friction surface curves away from said second friction surface forming an interaction portion and a recessed portion on said first friction surface, said recessed portion forming either a sharped-edged or a round edged groove on said first friction surface, having a depth and a tailoring width further away from said second friction surface.

7. The artificial bone joint of claim 6, wherein at least one portion of said second friction surface curves away from said first friction surface, forming an interaction portion and a recessed portion on said second friction surface, said recessed portion forming either a sharped-edged or a round edged groove on said first friction surface, having a depth and a tailoring width further away from said first friction surface.

8. The artificial bone joint of claim 6, wherein said depth of said recessed portion is of sub-micron in size.

9. The artificial joint according to claim 6, wherein said first friction surface is made of a metallic material, and said second friction surface is made of a resin material.

10. The artificial joint according to claim 6, wherein said recessed portion is adjusted in size as to said width and said depth for controlling wear particles generated from the said first and second friction surface.

11. The artificial joint according to claim 6, wherein the recessed portion is dimensioned such that wear particles generated from the first and second friction surface have a size or a shape by which the wear particles are excluded from a subject of a phagocytosis of macrophages.
